(19) United States
(12) Patent Application Publication
(10) Pub. No.: US 2003/0229452 A1
(43) Pub. Date: Dec. 11, 2003

(54) Multi-user system authoring, storing, using, and verifying animal information

(76) Inventors: Barrs S. Lewis, Minneapolis, MN (US); Tom M. Mayer, Mendota Heights, MN (US); John E.T. Shorrock, Minneapolis, MN (US)

Correspondence Address: Kagan Binder. PLLC, Maple Island Building, Suite 200, 221 Main Street North, Stillwater, MN, 55082, US

(21) Appl. No.: 10/340,944

(22) Filed: Jan. 13, 2003

Related U.S. Application Data
(60) Provisional application No. 60/348,768, filed on Jan. 14, 2002.

(51) Int. Cl. G06F 17/60; G06F 19/00; G01N 33/48; G01N 33/50

(52) U.S. Cl. 702/19; 705/3

(57) ABSTRACT

Systems and methods for integrating, managing and using electronic and tangible data relating to animals, especially data corresponding to official documentation. A secure, centralized repository for storing animal characteristic information, owner information, health information, official status information and the like is provided that may be used by a multiplicity of different user classes. Tangible counterparts of the electronic data also are provided, including documentation as well as fixtures that may be attached to an animal. A unique animal identification code is stored in the database and preferably appears on the tangible counterparts. The code serves as a primary key with respect to an animal's electronic records and allows records to be easily associated with a particular animal.

This application claims priority from U.S. Provisional Application Serial No. 60/348,768, filed Jan. 14, 2002, incorporated herein by reference in its entirety.

### FIELD OF THE INVENTION

The present invention relates to systems and methods for integrating, managing and using electronic and tangible data relating to animals, especially data corresponding to official documentation. More specifically, the present invention provides a secure, centralized repository for storing animal characteristic information, owner information, health information, official status information and the like that may be used by a multiplicity of different user classes. Tangible counterparts of the electronic data also are provided, including documentation as well as fixtures that may be attached to an animal. A unique animal identification code is stored in the database and preferably appears on the tangible counterparts. The code serves as a primary key with respect to an animal's electronic records and allows records to be easily associated with a particular animal.

### BACKGROUND OF THE INVENTION

The pet population problem in the United States is large. There are 36 million households that have 1.7 dogs for a total of 61 million dogs. There are 28 million households that have 2.2 cats for a total of 61 million cats. This is a total pet population of 122 million dogs and cats. Each year approximately 20 million pets are bought or adopted, half of which are dogs and half cats. The total population is relatively stable due to natural deaths, accidents and various public and non-profit efforts to control over population.

In accordance with conventional practices, pet owners often need to present to third parties (both regulatory and non-regulatory) proof in the form of a “certificate” of the health status of their pet or a municipal license. Third parties include entities such as veterinarians, government authorities, kennels, groomers, airlines & other common carrier transportation providers, game wardens (especially out of state), and international border controls who often require veterinary certificates testifying to the health, reproductive and/or vaccination status of pets. Third parties also include police and animal control authorities that may require proof of a city pet license.

There are certain issuers of licenses and certificates that depend upon the authenticity of information from other independent professional or regulatory entities in order to issue a license or certificate. An example is a municipal license authority that requires proof of rabies vaccination or of the reproductive status of a pet prior to issuing a pet license. Another example is a “dangerous dog” license issued to a pet owner whose pet has received a dangerous behavior citation issued by a regulatory authority.

License and certificate documents often require special handling since they do not fit neatly within a billfold, purse or pocket. Typically, the documents are folded as a consequence. If folded to fit conveniently for carrying or otherwise carried by the holder, a traditional certificate or license often becomes worn or torn over time. Thus, traditional pet documents are susceptible to damage. Documents are also easily misplaced, often times being permanently lost. When pet owners lose original copies certificates and licenses the issuers/originators of the documents are asked to provide duplicate copies. This service imposes inconvenience and additional administrative effort upon both the document issuer/originator and the pet owner. It also imposes a time burden upon the owner as obtaining duplicates of official documents can be a laborious process.

Third parties with an interest in the veracity of the information displayed on a license or certificate often have a need for a means to conveniently and reliably verify such veracity. The crude document technology used today for pet licenses or certificates can be easily tampered with and forged using the variety of photocopying and computer printing technologies readily available to anyone with a fraudulent intent. To realize the benefit of a system to verify official document content for the pet world it would be necessary that the system be secure, reliable, inexpensive, as well as, easily and ubiquitously accessible. Such competing concerns have not been adequately served in the past, and the demand for such a system remains strong.

Managing animal information is not the only problem at issue. Lost pets are another. Twenty percent of all pets are lost each year. This translates to 24.4 million lost pets annually and is a very big number. Most are returned to the owner. However, one in four is delivered to an animal shelter, and of these the vast majority (85%) are killed by the shelter. Only one out of every five pets that is delivered to an animal shelter is either retrieved by the owner or adopted by another owner. Animal shelters kill more than 6 million pets each year.

Both those pets that are safely returned and those pets that are lost forever typically cause anxiety and/or annoyance for the owner. For most pet owner households, one or more members consider a pet a beloved member of the family. In these cases, the threatened loss of a pet causes acute anxiety and, if ultimately lost, intense grief.

While the loss of pets does not rank anywhere near the top ten social problems of our day, 24 million lost pets is a big number and the associated time spent “finding” and retrieving the pets is large. For taxpayers the cost of feeding, caring and disposing of 6 million pets each year is high. It would be desirable to have a better system by which lost pets can be more quickly returned to their homes with less economic impact upon society.

The high-mobility life-style of today's population is one factor contributing to the high incidence of lost pets, troublesome experiences in retrieving a lost pet and unnecessarily frequent permanent loss of a pet. There are four life-style aspects to this high mobility: (1) the great geographical mobility of today's population beyond neighborhood boundaries within extended metro areas; (2) the peripatetic schedules of today's families where there are often two working spouses, heavy adult work schedules and busy after-school children activities; (3) vacation or weekend trips beyond home metro areas; and (4) less connected neighborhoods in which neighbors are less likely to know each other, each other's pets and are less likely to be home to “identify” a lost pet.

High mobility translates into many automobile trips often accompanied by the family pet. Every stop along the way presents an opportunity for a accident in which a pet escapes from the automobile without notice and becomes lost in an unfamiliar place whether it is the Kmart or Target in the adjacent suburb, the weekend cabin, or the cross country vacation.

This high-mobility and peripatetic life-style create opportunity for pets to be lost beyond the boundaries of their neighborhood. This complicates the task of a “good Samaritan” returning a “found pet” to its owner. Even within the neighborhood, the less-connected nature of today's neighborhoods impedes speedy return of lost pets. Young pets (less than two years old) that escape from the “yard or house” or the car outside the neighborhood without identification tags are the most frequent “victims”.

For the vast majority of all pets the identification and retrieval system for lost pets is essentially the same as it was 100 years ago. One or more tags are attached to a pet's containment collar. A tag typically provides a phone number that a “good Samaritan” or animal control officer can call to report a lost pet or an address to which to return the pet. A “good Samaritan” can be a neighbor or passer-by who finds a “lost” pet. The pet owner may or may not be available to answer the call or to accept the pet. The phone number or address may or may not be current or adequate due to the owner's failure to update the ID and to high mobility lifestyles.

Yet, tag(s) may or may not be on the pet. The primary reasons identification tags are missing are that: (1) the ID tag was accidentally separated from the collar, (2) someone removed the collar, or (3) an ID tag was never provided.

Metal or plastic identification tags are attached to a collar using “S” or “split ring” connectors. These tags hang and dangle from the collar and can snag on patio decks or shrub/tree branches. Often this results in accidental removal of a tag from the collar.

The owner or someone else often remove collars with hanging tag(s) while the pet is in the house. These collars are removed for several reasons: (1) multiple tags jingle and jangle making annoying noise, especially irritable at night; (2) the tag(s) hangs down into the pet food and can ding loudly against the food bowl; (3) the tag(s) hangs down and is unsightly; (4) the hanging tag(s) scuffs and marks furniture and (5) the containment collar to which a dog's tag(s) is attached is often removed and interchanged with a variety of other containment collars that are used for different purposes such as a trainer or choke collar. Although removed for a variety of reasons the result is the same. Pets can escape from the house or an enclosed yard when a door is opened and the pet springs out or left open unattended and the pet simply leaves.

Typically in the United States three separate identification tags are attached to the collar when owners fully protect their pet with proper identification and comply with local animal control laws. The three tags are: (1) a rabies tag, (2) a municipal license tag and (3) an identification tag that usually displays some combination of a name, telephone number and owner address. Most owners comply with the rabies tag requirements. It is reported that fewer than 10 percent of all owners comply with the municipal tag requirement. A significant number of owners display no effective identification tag (sufficient and accurate information) on their pet's collar. In some countries such as the United Kingdom rabies is not required but identification is required by law.

Children and others also remove collars with attached ID tags without the consent of the “owner”. The reasons can vary. Playing or washing are probably the most prevalent. The result is the same. The pet can be lost without a collar with attached identification tags.

Finally, some owners simply don't attach ID tags to their pet. The reasons vary from dislike of fur discoloration that can be caused by aluminum tags, to the dislike of the “hanging” tags for all the reasons previously mentioned, or to simple procrastination and negligence. The most significant of these is the last and it is very significant.

It is easy not to buy a primary identification tag. Tags are a small and insignificant revenue item at a pet store ($4.00-8.00). At the same time, tags require a significant amount of bothersome customer service and follow-up due to their need to be customized to display information unique to each pet. Tags also have very low spontaneous consumer appeal. They are viewed as utilitarian and “good for the pet” but ID tags simply don't have much merchandise appeal. Consequently, ID tags are not prominently displayed and promoted at the point of sale.

When tags are purchased at the retail point-of-sale the responsibility to complete the registration is typically left to the consumer. The consumer must “close the loop” or follow-up by mailing information to the tag supplier who sends the tag to the consumer with the requested information or by engraving the ID information on the tag using a consumer operated engraving machine located at the store. The problems with the latter is that it is time consuming, a bother, something that can easily be delayed until the “next” visit and only high volume stores can afford the fixed cost associated with the engraving machine. Most identification systems sold at the retail point of sale require a mail interaction with the tag supplier.

A couple of significant things must happen here. First, the consumer must (1) complete and (2) return the information to the tag company. Second, the pet owner must attach the tag to the pet's collar when the tag is received in the mail from the supplier. Neither of these two steps requires rocket science intelligence or great effort to execute. Nonetheless, completing both steps is a compound event that defies human nature and good intentions for most people.

The phenomenon is similar to the well-known phenomenon in the use of manufacturer rebates in the field of promotional marketing. A manufacturer offers manufacturer rebates contingent on the consumer mailing a rebate coupon and proof of purchase to the manufacturer following purchase of the product. Rebates influence purchase decisions at the point-of-sale, but most (over 93%) consumers don't follow-through with return of the rebate and proof of purchase.

Similar behavior is known to be true for pet identification tags. The city of Minneapolis Animal Shelter introduced microchip identification technology in the early 1990's. When initially introduced pets that were adopted or retrieved from the animal shelter were automatically injected with the very reliable microchip identification technology at the time of adoption. The owner paid a relatively substantial amount ($50.00) for this and was provided a form to complete and mail to a national online retrieval registry maintained by the American Kennel Club. Fewer that 10 percent of the new owners followed through with the ID registration. They had already paid for the service and the pet had already been injected. Yet, procrastination and human nature prevailed in 90% of the cases; pet ID registration was not completed.

This is both an amazing and significant statistic in understanding a huge hole in today's pet identification system. In system engineering terms, there is an “open loop” that results in failure of the system to perform properly. The “open loop” aspect of pet identification systems has profound implications on the inadequacies of today's solution and the requirements for implementing a superior solution.

Two forms or permanent identification are available on the market that are intended to provide a much more reliable identification technology that will prevent the detachment of an identification tag from a pet. These are: (1) tattoo and (2) injected microchip. Roughly 6 million of all pets have tattoos and one million have microchips. Pet tattoos have been in use for 30 years; microchips for 10 years. Both technologies are a response to the recognized inadequacies of the dangling tag attached to a collar. Both technologies have strengths but both have significant limitations.

Both technologies share advantages. They are: (1) permanent and solve the unreliability problem of tags being detached from collars or collars being removed; (2) are associated with central registries; and (3) offer a “closed loop” solution to completing the initial registration process. Both technologies also share the common disadvantages: (1) the identification procedure is invasive, it temporarily “hurts” the pet; (2) the procedure requires an appointment with a specialist, vet in the case of a microchip and groomer or vet in the case of a tattoo; and (3) the procedure is expensive, $30-$50.

Typically, the professional service provider who applies a permanent ID to a pet takes responsibility for completion of the registration process. This greatly improves the reliability of the over-all ID service.

Each technology has a disadvantage unique to itself. Tattoos have three significant disadvantages as an identification technology for aiding in the return of lost pets. First, tattoos are first and foremost an animal marking technology for breeder purposes to establish ownership and lineage. There is no standard for tattoos nor is there a central registry. Instead there are numerous (more than 30) tattoo registries maintained by different breeders or associations of breeders. Similarly, there is no visible telephone number to call to assist in identifying and notifying the owner. Second, tattoos typically require “handling” the pet to find and read the ID. Strangers are reluctant to handle a “strange” animal. This is not a problem for animal control or health care professionals but it is a significant obstacle to the easy casual visual identification of a pet by a “good Samaritan”. Third, the tattoo can also fade as the pet grows or the fur changes color.

Microchips provide no utility as a visual identification technology since it is injected under the skin of the pet and is therefore invisible. Microchips require an electronic reader to read the ID. These readers are available in a growing number of animal shelters and vet clinics, but the necessary readers are not universal among professionals and are generally unavailable for use by the general public. A “good Samaritan” certainly wouldn't be expected to have a reader. This means a “good Samaritan” must deliver the “lost pet” to a vet clinic (with a proper reader) or to an animal shelter (with a proper reader).

On the other hand, microchips have the significant advantage of providing an identification of “last resort”. Many animal control shelters and most metropolitan animal shelters have microchip readers that provide an efficient means without handling the pet to determine if the pet has a microchip. Most pets are “read” immediately prior to being put-down (euthanasia) to determine if the pet has a microchip. Pets injected with a microchip are unlikely to loose a pet delivered to the shelter, if the associated registry information is both current and sufficient to contact the owner.

The same shelters equipped with the microchip technology automatically microchip pets delivered to the shelter before releasing for adoption. However, the fact is most pets don't have a microchip. Although both technologies address three of the most significant limitations or the collar tag technology, the disadvantages are sufficient to have prevented significant market penetration. Distribution channel convenience, costs and invasive procedure are significant obstacles.

The first step in retrieval of a pet displaying an identification tag is to notify the owner or a representative thereof that a “lost” pet has been found. There are several reasons why owner notification systems are cumbersome and time consuming or fail altogether. They are: (1) information is provided on the ID tag for contacting the owner or a representative but “no one answers” the phone or the door; or (2) information on the tag is wrong or incomplete.

In a perfect world a “good Samaritan” would look at the telephone number, name of the pet and owner and the address of the owner and with the use of this information decide whether to return the pet to the owner's home or to a nearby shelter or vet clinic. In this ideal world, the owner (or a related party) would be home to accept the phone call and/or the vet clinic would be open for business.

In the real world, tags often have insufficient information for easy, quick and reliable return of a lost pet. Due to their small size, tags are severely restricted in the amount of information that they display. With a population in perpetual motion, one phone number is often insufficient to make a real-time connection with an owner. The chances of success go way up with additional numbers or contacts. Today's tag technology simply can't provide the type information to make notification quick, easy and reliable.

Furthermore, in the real world even if a visual identification tag provides the necessary information, the telephone often is unanswered by a live person, an answering machine perhaps, but not a live person. In the case of the pet owner, the typical American's highly mobile peripatetic life style is the culprit. Mobility beyond metropolitan boundaries to weekend cabins and car vacations create even more severe obstacles to notifying the owner when a lost pet is found. People aren't home and an answering machine often isn't the solution. It is a lot to expect that a “good Samaritan” will invest significant effort to contact an owner with whom they have no personal relationship. The “good Samaritan” will invest “some” effort, but typically not a lot of effort.

The neighborhood vet clinic is a good choice if: (1) the clinic is not closed for the night or weekend and (2) the “good Samaritan” is willing to load the pet into his/her car and deliver it to the vet. Police are an option as are 24-hour animal shelters. The inability to easily and quickly contact a human who will take responsibility for the situation is a big failure in today's notification system.

The high mobility of today's population results in inevitable changes in address and phone number. The likelihood of updating ID tag information is typically less than the chance of successful initial implementation of a new ID tag. Outdated and inaccurate owner contact information leads to a direct failure of the efforts of a “good Samaritan” to notify an owner of a lost pet that has been found.

Central registries provide a potential means to overcome some of the shortcomings that cause owner notification efforts to fail. A central registry provides a number to call where an operator can provide immediate information that may be helpful in notifying a pet owner. Registries are often available 24 hours a day; many registries such as those provided by vet clinics, animal shelters and municipal license bureaus are not. Registries are often limited in the information they provide.

Today, there exist numerous pet registration databases: city license agencies, veterinary clinics, breeder registries, and others. The largest two registries for breeders are American Kennel Association Club (that sponsors microchip registration) and the National Dog Registry (that sponsors tattoos). The more than 30 breeder databases suffer from fragmentation, access hours that are often restricted, and outdated information. The city license offices are often very difficult to contact and have limited office hours.

The accuracy of registry information is often poor for two reasons: (1) ID tag information never reaches the registry following purchase of a registered identification system, (2) registry information is not updated when a pet owner temporarily or permanently changes address, telephone number or other pertinent information. Both are significant drawbacks. This failure in today's system is due to the same tendency toward procrastination and negligence that plagues completion of the initial ID implementation process. The particular example of the failure of new pet owners to follow-through on the registry of microchip implants was previously discussed. Ninety percent of all pet owners failed to mail the microchip registration form even though the microchip injection was already performed and paid.

As is the case with ID tags, the likelihood of updating information is typically less than the chance of successful initial implementation of a new ID tag or pet identification registry. In both cases, outdated and inaccurate owner contact information leads to a direct failure of the efforts of a “good Samaritan” to notify an owner of a lost pet that has been found.

Increased mobility of the population has contributed considerably to the number of lost pets. When pets are lost out of their neighborhoods the owner is usually left to search frantically on their own or with friends. This is usually taking place in non-familiar territory and so makes the search harder. Under these unfamiliar circumstances it is hard to get quick access to search and recovery services to help with searching the neighborhood.

There are times when proof of ownership becomes an issue in reuniting pets with their owners. Here microchip or tattoo animals will eliminate this doubt. However many animals are not micro chipped or tattooed and so other proof is necessary. There are cases where finders convince themselves that pets have been abandoned or simply keep the pet. Shelters and vets also require proof of ownership and vaccinations for lost pets. At this time there are no good methods other than microchips or tattoos for proving ownership.

The retrieval process for lost pets today has many “open loops” and other inefficiencies due to fragmented databases, often poor and unreliable pet identification aides, and life style patterns that often complicate, delay and/or prevent the return of a pet to its owner. The result is either: (1) prolonged (longer than necessary) anxiety and or inconvenience to the pet owner or (2) permanent loss of the pet and the associated loss of a member of the “family”.

There is no question that the reliability and speed of animal retrieval can be significantly improved by a solution that addressed the obstacles described herein. To be effective, the solution must be designed and operated as an integrated service solution that addresses all of the major obstacles. To successfully implement an improved solution into the broad consumer pet market, the solution must tap an effective and efficient distribution channel. A solution that reduces the time and inconvenience for the return of a lost pet would tap an unfulfilled market demand for an improved solution.

### SUMMARY OF THE INVENTION

The present invention provides a service system network for storing, using, and managing information about animals, particularly pets. An important element of the invention is a centralized electronic repository in which animal data, particularly electronic counterparts of official documentation, is securely stored and readily accessible to different classes of users. The emergence of the Internet, online database server technology, distributed client/web software methods and software security and authentication methodologies have enabled the opportunity to design and develop new methods and systems for certifying the health and regulatory license status of pets. The system of the present invention does not rely solely upon paper documents that alone are cumbersome to carry, easily destroyed, and easily susceptible to fraudulent tampering.

Through the use of online databases, Internet communications, interactive software application methods, and optionally any of a variety of data encryption methods (e.g., radio frequency identification (“RFID”) technology, magnetic and bar encoding systems, and credit card material technology) it is possible to provide a reliable online information service that can be reliably used by multiple parties including: (1) professional and regulatory entities that issue or originate official certificates and licenses, (2) pet owners who must present documentary proof of the certified or licensed status of a pet; and (3) third parties who require knowledge of the health and/or vaccination status of a pet certified by a veterinarian professional to be true.

The system offers a variety of advantages to pet owners, veterinarians, municipalities, airlines, customs/border officials, groomers, police, animal control agents, kennel employees, and pets. Firstly, the present invention offers: (1) secure, easy and reliable entry by originating/issuing authorities of official documentary data into a central database; (2) display of official document data in a variety of methods including but not limited to: (i) a durable, tamper resistant, compact and easy-to-carry card that may be carried by a pet owner or custodian, (ii) a printed paper reproduction of the electronic form of an official document and (iii) an electronic display of the electronic form of the document; and (3) easy and reliable verification methodologies for third party entities that may desire to confirm the veracity of a license or certificate.

The present invention also offers secure methods for pet owners to create, modify, use, and securely store information in the database that is not of an official nature. Such information may be of two types: (1) mandatory information such as name, address and minimum contact data and (2) optional data such as additional contact information and information pertaining to the physical and behavioral status of the pet where such information is not information that is certified by a veterinarian professional.

The invention also allows electronic counterparts of parent and dependent documents to be linked together. Additionally, a verification system provides secure and convenient methods for an entity such as a municipal licensing entity to obtain, verify and link the issuance of a license to the electronic form of a certificate issued by a licensed professional (veterinarian) that certifies the health status of a licensed pet. The practical application of this novelty is to link an electronic certification by a licensed veterinarian of the vaccination (rabies) and reproductive status (spayed or neutered) of a pet to the municipal licensing authority that licenses pets based on proof of the rabies vaccination and reproductive status of a pet. Typically, proof of a current rabies vaccination is a requirement for issuance of a municipal pet license whereas proof of the reproductive status of the pet determines the amount of the license fee. Other entities may also verify animal information presented to them using the system.

The system enables authorities to designate agents to create and issue official documents or electronic counterparts thereof on behalf of the authority through secure processes that assure compliance with the regulations prescribed by the authority. Audit trails and electronic reports of activity performed by the agent may be provided to the authority to verify compliance.

The system also optionally integrates methods for billing end-user subscribers (pet owners) for the creation of official documents and the remittance of license and certificate fees to the originating or issuing entity. Billing and payment methods may include electronic commerce technologies.

Another key advantage of the system is that it provides a methodology that can facilitate fast, easy, and reliable return of a lost pet to its owner so that the anxiety and annoyance associated with losing a pet is as short as possible. First, the system provides easy convenient and reliable means for entry of and access to information on how to locate the owner for use by a “good Samaritan” who finds and attempts to return a “lost” pet. Second, the system provides an automated and proactive search and recovery tools that are easily and readily available to a pet owner who is aware that a pet has been lost and chooses to initiate action to seek its recovery.

Search and recovery capability includes the capability to send electronic alerts, identification (picture and description) information and contact information to entities that might be instrumental in the recovery of the lost pet. Examples include email and fax transmission to veterinary clinics, animal control agencies, police, and individuals in the vicinity of where the pet was believed to have been lost. Another example is provide an easy directory containing the names, addresses and phone numbers of these same entities to the pet owner so that the owner can contact the entity directly to seek assistance.

The different aspects of the invention are defined in the independent claims provided below.

### BRIEF DESCRIPTION OF THE DRAWINGS

The above mentioned and other advantages of the present invention, and the manner of attaining them, will become more apparent and the invention itself will be better understood by reference to the following description of the embodiments of the invention taken in conjunction with the accompanying drawings, wherein:

FIG. 1 is a schematic diagram showing one embodiment of different categories of content that may be stored in the electronic database of the present invention.

FIG. 2 is a schematic diagram showing the relationship between the database of the present invention and different users.

FIG. 3 is a schematic diagram more concretely showing the relationship between the database of the present invention and specific classes of users.

FIG. 4 is a schematic diagram of one embodiment of a record structure of the present invention showing how a preferred record comprises content of one or more fields and a profile of one or more properties.

FIG. 5 is a schematic illustration of one situation in which multiple records of the present invention are linked together in a parent/dependent relationship.

FIG. 6 is an illustration of one embodiment of a data entry form by which a user may input owner information into the database of the present invention.

FIG. 7a is an illustration of one embodiment of a general query form that a user may use to locate information stored in the database.

FIG. 7b is an illustration of the general query form of FIG. 7a filled out with specific query information.

FIG. 7c is an illustration of the general query form of FIG. 7a filled out with specific query information.

FIG. 7d is an illustration of the general query form of FIG. 7a filled out with specific query information.

FIG. 8a is an illustration showing one format in which search results resulting from the query of FIG. 7b may be presented.

FIG. 8b is an illustration showing one format in which search results resulting from the query of FIG. 7c may be presented.

FIG. 8c is an illustration showing one format in which search results resulting from the query of FIG. 7d may be presented.

FIG. 9 is an illustration showing one major face of a preferred pet identification card of the present invention.

FIG. 10 is an illustration showing a second major face of the pet identification card of FIG. 9.

FIG. 11 is a schematic diagram showing one approach by which the system of the present invention may be used in connection with an animal vaccination.

FIG. 12 is a schematic diagram showing one approach by which the system of the present invention may be used in connection with the issuance of a municipal animal license.

FIG. 13a is a schematic diagram showing one approach by a “good Samaritan” to use the system of the present invention to return a lost pet to its owner.

FIG. 13b is a schematic diagram showing one approach for a pet owner of a “lost” pet to initiate a search and recovery process by contacting entities who might find the pet and notify the pet owner.

FIG. 14 is a schematic diagram of a preferred system of the present invention that facilitates and search and recovery initiative on behalf of and by the pet owner.

FIG. 15 is a schematic diagram of a preferred system architecture of the present invention.

FIG. 16 defines the arrows used to schematically show interaction among software components in FIGS. 17 to 23.

FIG. 17 is schematic diagram of a preferred registration subsystem.

FIG. 18 is a schematic diagram of a preferred medical history subsystem.

FIG. 19 is schematic diagram of a preferred certificate creation subgroup of a certificate and license creation subsystem.

FIG. 20 is schematic diagram of a preferred license creation subgroup of a certificate and license creation subsystem.

FIG. 21 is schematic diagram of a preferred certificate and license access subsystem.

FIG. 22 is schematic diagram of a preferred search and recovery subsystem.

FIG. 23 is schematic diagram of a preferred appointment scheduling subsystem.

### DETAILED DESCRIPTION OF PRESENTLY PREFERRED EMBODIMENTS

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention.

The present invention provides an integrated system for managing and using animal information in both electronic and tangible form. In preferred aspects, the system may be used to help promote the general health and well being of any animals, but livestock and/or pets in particular. The preferred system includes (1) a centralized database that is used as an electronic repository of data for a plurality of animals, (2) tangible documentation that comprises information that matches data stored in the database, and (3) tangible fixtures or elements (e.g., tags, studs, tattoos, clips, embedded or implanted devices, stickers, etc.) that are attachable directly or indirectly to an animal and that also comprise information that matches data stored in the database. Most preferably, a unique animal identification code is associated with each animal and may be used to associate the electronic and tangible information to the corresponding animal.

The system preferably provides methods for (1) communicating with the database to create, edit, access, generate reports/documents, or otherwise use the stored data; (2) displaying data in a tangible form, preferably on fixtures and/or documentation such as a durable, convenient to carry, and tamper-resistant card; (3) displaying the content of official data in a tangible form, e.g. in the form of documents on paper, on the card noted above, on attachable fixtures, or the like (4) displaying official records or documents/reports generated therefrom on electronic devices; (5) allowing inquiring third parties to verify remotely that the tangible form of the official data is the same as the electronic form stored in the database; (6) providing a vehicle by which “good Samaritans” may locate pet owners and return lost pets to their homes; (7) providing automated electronic means for a pet owner to initiate a search and recovery activity; (8) allowing pet owners to append and modify appropriate database information about their pets; (9) allowing authorized entities to create electronic versions of official data and store those in the database; and (10) allowing third parties to access the database and verify the health status, legal status, or other information relating to an animal.

The database preferably uses security protocols to assure that (1) particular kinds of data can be created, modified, accessed, or otherwise used only in appropriate circumstances by appropriate personnel (such as limiting the circumstances under which data will be generated or by restricting the individuals who have the authority to use certain kinds of data); (2) certain kinds of data can be accessed and manipulated, e.g., via report generation, by individuals or database functions having authority to do so; and (3) certain kinds of data stored in the database is transmitted and retained in authenticated form.

A preferred set of security protocols will achieve one or more desired objectives. First, such protocols will control who can create and edit records. This may be accomplished using a variety of security tactics. These include requiring most users (a “good Samaritan” could be an exception) to enter a name and password before being allowed access to the database. The system may then offer a user access rights depending upon factors including the class to which the user belongs and the subscription service for which the user registered. For example, veterinarians, pet owners, municipalities, and “good Samaritans” each represent a different class of user, and each may be accorded different access rights to the database. The database may also require any user to complete a registration process before the system will assign a user name and password to get user.

System security may also associate data into different kinds of classes such that data class is a factor affecting access rights to particular data in the database. Thus, pet owner information, pet characteristics, veterinarian information, clinic information, “good Samaritan” data, official data, and the like represent different classes of data for which access rights may respectively vary. The system security may also control who can generate tangible output from the database. For example, only certain authorized entities may have the authority to create, access, update, and/or generate tangible copies of municipal licenses, rabies tags, or the like. Like many financial software programs, the system security may also maintain a history of all actions taken on the database, how the actions were taken, and who took those actions so that use of the database can be audited when desired. In the case of official records corresponding to tangible, official documentation generated under the purview of a governmental authority, the system security may include procedures so that such official output when generated is authenticated. A variety of security protocols for accomplishing these objectives are known and any conventional, effective protocols may be used. See, e.g., U.S. Pat. No. 5,657,390, which describes SSL from Netscape. Another example is RFC 2246 by IETF (Internet Engineering Task Force) for TLS, which is an open alternative to SSL.

The electronic form of the data allows the data to be securely stored in a centralized repository and accessed on demand by a variety of users. The purpose of the tangible form of data is to provide an animal owner or custodian, as the case may be, with a means to easily and conveniently present animal information to third parties who may have a desire or need to know the legal status or other information pertaining to the animal. Examples of third parties who may review such information before rendering services relating to an animal include a wide variety of entities such as groomers, animal show administrators, kennels, airlines, customs and border control authorities, veterinarians, and the like.

The tangible counterparts of data stored in the database most commonly include information relating to health, vaccinations, and licenses. For example, data relating to a rabies vaccination, such as data about the vaccination itself or data corresponding to a rabies certificate, may be stored in the database electronically. In the meantime, a corresponding, tangible rabies fixture may be provided and then directly or indirectly attached to the corresponding animal. A paper copy of a rabies vaccination certificate may also be carried by the animal owner or custodian, as the case may be. Information included in such tangible counterparts advantageously may include data that may be used as a link to the database system so that electronic data stored in the system can be used to confirm the veracity of information contained on the tangible items. Alternatively, in the absence of any tangible counterparts, electronic data stored in the system can be used to determine the status of an animal independent of any tangible items in the event that such items have been misplaced or are otherwise unavailable.

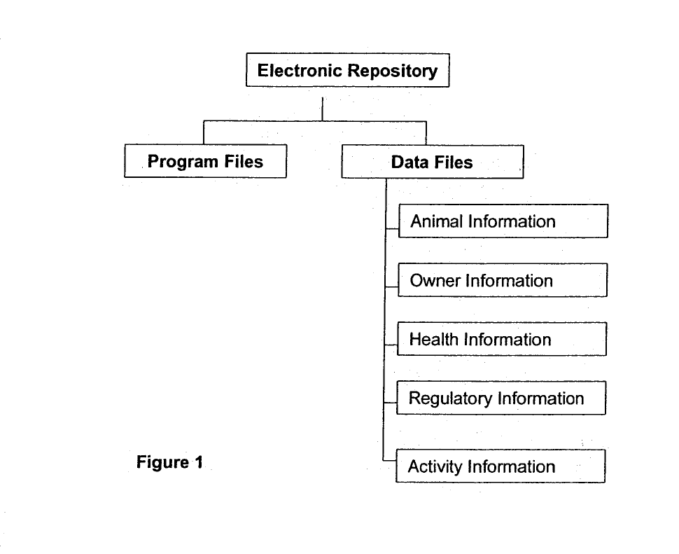

FIG. 1 is a schematic diagram that shows the content of a preferred electronic repository of animal information of the present invention. FIG. 1 shows that a wide variety of different kinds of animal information in one or more different categories can be maintained in the database. Representative examples of such information include animal characteristic data, animal owner data, animal health data, regulatory data, “good Samaritan” data, animal activity data, and/or the like.

Animal characteristic data generally includes information that helps to identify a particular animal. Such information may include the name, breed, gender, weight, height, eye color, distinctive markings, reproductive status, birth date, photograph, and the like for an animal.

Animal owner data generally includes information that helps to identify the owner of a particular animal. Animal owner information preferably includes for example name(s), residence address(es), phone number(s), e-mail address(es), drivers license number(s), and the like for the one or more parties that own or are otherwise responsible for an animal.

Animal health data generally includes information indicative of the health and health history of an animal. Such information may include general health status information, diet information, vaccination information, veterinarian clinic information, and the like. General health status information includes information such as reproductive status, behavior characteristics, health treatment information, prescription information, and the like. The vaccination information preferably includes vaccination information for rabies, distemper, bordetellosis, para influenza, hepatitis, and/or any other condition for which a vaccination is administered. For each such vaccination, detailed vaccination data might include the entity that administered the vaccination, the date on which the vaccination was administered, the expiration date of the period in which the vaccination is current, and identification of the vaccination that was administered (e.g., manufacturer, brand-name, dosage, Lot No., and the like). The veterinarian clinic information preferably includes the clinic name, doctor name, clinic address, clinic phone number, electronic signature of veterinarian, professional license number of veterinarian, and the like.

Regulatory information includes information about the applicable regulatory jurisdiction(s) as well as official data applicable to a particular animal. As used herein, “official” in connection with data means that the data was created under the purview of a governmental authority or licensed veterinarian professional and that the data indicates a legal status for an animal. Generally, only governmental entities or authorized agents thereof have authority to create such data. Official data may be in electronic or tangible form. Examples of official data include municipal licenses, vaccination certificates, vaccination tags, license tags, dangerous animal licenses, and the like. While the kind of regulatory information for animals may vary from jurisdiction to jurisdiction, most animals are required to have one or more of a vaccination certificate or municipal license.

In the practice of the present invention, electronic counterparts of conventional animal licenses, rabies certificate, and the like may be readily stored and accessed on demand from the database of the present invention. The centralized, electronic storage approach advantageously reduces the likelihood that important documents will be lost, destroyed, fraudulently transferred or fraudulently presented to an inquiring third party. Access is much easier than if documents are stored somewhere in a file in paper media. Much less storage space is needed to store electronic data as compared to tangible counterparts of such data.

A governmental authority having primary responsibility for the creation and issuance of official documents can designate a third party entity to act as its “agent”. The agent can be authorized to create and issue official documents on behalf of the authority. An example of this is the case where a municipal government authorizes a particular veterinarian clinic to issue municipal pet licenses on behalf of the municipal government, subject to applicable legal requirements. The system preferably would allow such a governmental authority to register one or more agents using a suitable registration process. Once an agent has been designated and established in the database, the agency may create and issue of official documents so long as it is empowered to do so.

Animal activity data may be created and stored to document the participation of the animal in various events, such as training, animal shows, breed registration, animal competition, breeding, work tasks, and the like. In a fashion, the animal activity data may serve as an electronic resume for the animal.

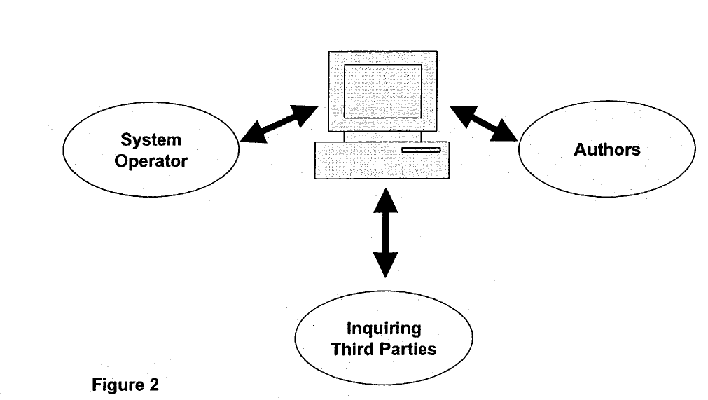

FIG. 2 shows the general relationship between the database of the present invention and various entities that may wish to access, create, and use the database. Authors are those entities who create, edit, or add data to the database. The system operator represents the service provider(s) that provide many database management tasks, such as handling administration of the database, maintaining the database, and providing customer service to help others to use the database. Inquiring third parties represent entities that access the system to determine information about an animal but generally do not modify any information in the database in connection with such access except in limited, restricted circumstances. In some circumstances, an inquiring third party may have the authority to generate documents and/or reports from data stored in the database. The same entity may act as an author in one context, but as an inquiring third party in another context.

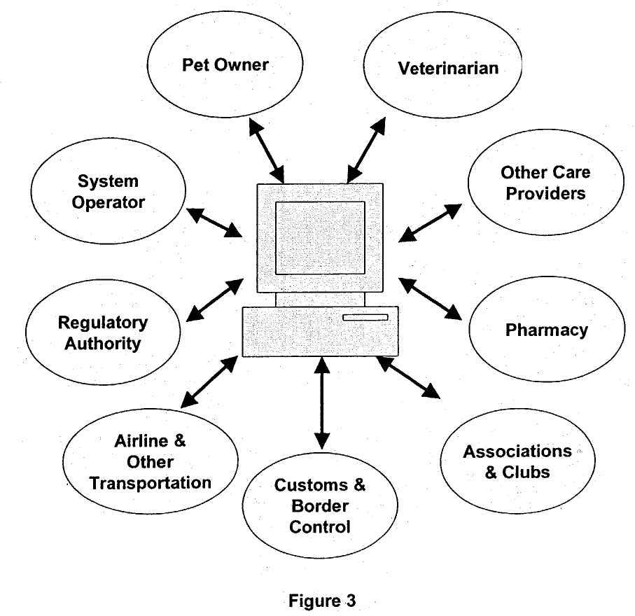

FIG. 3 shows the relationship between various specific entities and the database in one preferred aspect of the invention. The system operator is present to help administer the database, maintain the database, help others to use the database, etc. The pet owner in one set of circumstances may be an author who desires to store information about himself/herself and about his/her pet. The pet owner may create, update or the add data to the database relating to owner information, pet characteristic information, lost pet information, or the like. In other circumstances, e.g., when wishing to confirm the health or regulatory status of the pet, the owner may also interact with the database as an inquiring third party.

A veterinarian may also interact with the database in many respects. In some circumstances, after treating an animal and/or administering a vaccination, the veterinarian may function as an author by accessing the database, creating new health/vaccination data, and storing it in the database. Under appropriate authority of a regulatory entity, e.g., a state government, municipal government, or the like, the veterinarian may also create and store electronic data counterparts of official documents such as vaccination certificates and/or animal licenses in the database. A veterinarian may create an electronic form of a pharmaceutical subscription that can be accessed by a pharmacy. In other circumstances a veterinarian may act as an inquiring third party when merely querying the database to learn information about an animal in connection with a treatment or other service.

The regulatory authorities may act as authors when creating and storing official animal information, e.g., vaccination certificates data, licenses data, or the like, on the database. In other circumstances, a regulatory agency may query the database to determine the status of a particular animal in circumstances where animal status or behavior creates an issue within the jurisdiction of the agency.

Many times, a variety of third parties may need to determine the health or regulatory status of an animal before taking certain action with respect to the animal. For example, a groomer or kennel may wish to confirm the health or regulatory status of an animal before providing a grooming or kennel service. A pharmacy may confirm or obtain subscription information created by a veterinarian. A transportation service such as an airline may need to confirm the health or regulatory status of an animal before allowing the animal to be transported. Customs/border control authorities may also wish to confirm the health or regulatory status before allowing an animal to be transported from one territory to another. In all such circumstances, a groomer, kennel, transportation service, customs/border control, or like may query the database as an inquiring third party in order to learn the desired information about an animal.

Thus, it can be appreciated that the present invention provides a secure, remote, centralized verification database system that can be used by issuers and originators of official documents to create and store official information about an animal; by pet owners who might need to display information about their pet to third parties: and by third parties who may wish to verify information about an animal in a variety of circumstances. The system of the present invention is most advantageously used as a centralized, remote repository for official documents for animals that may include but are not limited to vaccination certificates government regulated licenses (including municipal, dangerous animal, competitive, and other licenses), ownership certificates, breeder registry certificates, and the like. The system also offers easily and ubiquitously accessed means for third parties to determine and verify information about an animal. In preferred embodiments, the system also includes optional procedures by which a “good Samaritan” who locates a lost pet can access the system in order to directly or indirectly contact the pet owner so that the lost pet can return to its home.

The database of the present invention is likely to store animal information for a plurality of animals, preferably thousands and thousands of animals. In view of the large number of animals for which animal information could be stored in the database, it is important that data is easily associated with particular animal(s) when being created, edited, accessed, or otherwise used. In the present invention, data is generally associated with a particular animal, owner, veterinarian clinic, or other entity by using a suitable primary key. In the practice of the present invention, the primary key preferably is in the form of a unique animal identification code that assures that each and every animal having information stored in the database has its own, unique identification code distinguishing it from other animals in the database. The unique, animal identification code is analogous to the social security number for a human being.

The unique animal identification code may be any unique symbol or series of symbols. The unique animal identification code is desirably in a format that can be represented in both machine readable and human readable form. This allows tangible counterparts of the identification code to be visually discerned by a party or, if close contact with an animal is desirably avoided, the machine readable form can be read from a safe distance using a suitable interrogation device. Accordingly, codes that comprise a sequence of alphanumeric digits are most preferred. Each individual digit of an alphanumeric code can either be a number from 0 to 9 or a letter from A to Z. The number of digits constituting the code determines how many different codes may be used. In the practice of the present invention, codes comprising seven to fifteen digits are preferred to ensure that there are enough different codes to satisfy demand.

When creating an electronic data record for a particular animal, the data is easily associated with the animal simply by associating the data with the animal's corresponding code(s). This association may be accomplished electronically by including the animal identification code as an actual field in the data record. Alternatively, the electronic data record may be associated with the code by a suitable link. Similarly, when looking for a particular record for animal, e.g., rabies vaccination status, the user can query the database to locate the particular rabies vaccination status record that corresponds to a particular animal identification code. Just as easily, all records for a particular animal are easily located simply by querying the database to locate all records associated with a particular animal identification code.

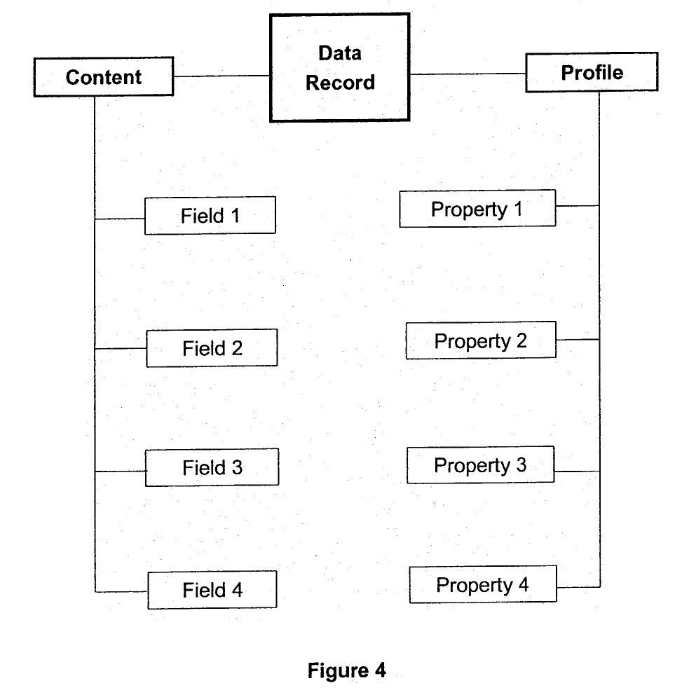

Animal data may be stored in the database in any suitable fashion. In preferred embodiments, data is stored in the form of data records such that a plurality of animal information records for a plurality of animals are stored in the database. Each record may be associated with an animal by information comprising the unique animal identification code corresponding to that animal. As shown in FIG. 4, preferred records of the present invention generally comprise content that includes one or more fields of information, and a record profile that includes one or more record properties. At least one of such record properties is desirably the unique animal identification code that associates the record with a particular animal. Record properties are particularly helpful in order to create, store, locate, modify, or generate documents or reports from stored data records.

In terms of content, the particular field or fields for a particular record will vary depending upon the class of record. The class to which the author belongs may also affect the fields that are available. Thus, a record for owner information will likely comprise different fields of information than a record corresponding to an official document such as a municipal license or rabies vaccination certificate. Likewise, an owner record may appear differently to the owner than it does to a third party.

On the other hand, the record properties may be substantially uniform and even identical from record to record. A record property comprising a unique animal identification code is helpful for associating records with a particular animal. Many other record properties also are useful for working with records of the database. Representative examples of other useful properties include, for example, a unique record number that the system automatically assigns to the record, a document title, a record class (e.g. health record, pet information record, owner information record, official license, official vaccination certificate, and the like), the author of the record, a class to which the author belongs (e.g., pet owner, veterinarian, municipal authority, or the like), creation date, last edit date, expiration date (if applicable), link(s) to any parent records, link(s) to any dependent records, applicable security, verified or official status, and the like.

Data records may be stored in the database in any suitable fashion and in accordance with conventional practices. A variety of other database structures are known and usable in the practice of the present invention. Examples are commercially available from vendors including Oracle, Sybase, Microsoft, IBM, Informix, and the like.

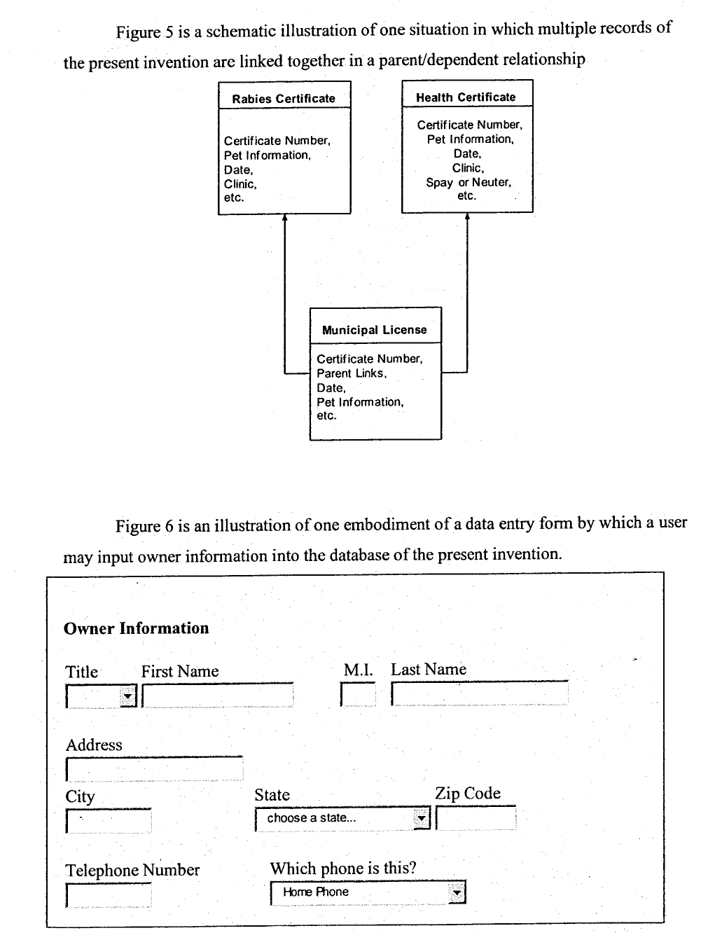

In some instances, the issuance or existence of animal information may depend upon the existence of other information. This typically occurs, but is not limited to, situations in which the issuance of a municipal license or vaccination certificate is dependent upon the health and/or ownership status of a pet. FIG. 5 is a specific example of this situation. In FIG. 5 the municipal license or electronic counterpart thereof for a particular animal is issued only if the animal has a current rabies vaccination certificate or electronic counterpart thereof. The rabies vaccination certificate, in turn, is issued only if the animal has been vaccinated for rabies. Thus, the municipal license, the rabies certificate, and the rabies vaccination treatment are linked together in the sense that the license and/or certificate may be created using information from one or more other data items. Once created, each such license or certificate, as the case may be, preferably exists on its own merit independent of other document(s) used as a source to create all or a portion of the license or certificate. Optionally, however, the system may include functionality to determine underlying data sources from which a certificate or license was created.

Advantageously, the present invention allows dependent and parent records to be easily linked so that the relationship of such linked records (i.e. the dependent or parent) can be readily determined. The database may also be queried to quickly locate such linked records. There are many practical circumstances in which such linking is desirable. For example, before issuing a rabies vaccination certificate or a municipal license, or the like, a regulatory authority may wish to confirm that any prerequisites, the e.g., the existence of a current vaccination certificate, has been satisfied before issuing an official license or electronic counterpart thereof. In the practice of the present invention, this is easily done by accessing the database, searching the database for appropriate records corresponding to the animal, and confirming whether the prerequisites have been satisfied.

To facilitate electronically linking parent and dependent records together, it is preferred that the profile for records of the database include one or more profile properties allowing linked documents to be readily identified. For example, in embodiments of the present invention in which each stored record of the database is assigned a unique record number, the profile properties that identify a link might include the unique record number and relationship (e.g., parent or dependent) of the record to be linked. When one record is linked to another, the record profile properties of each generally may be modified to include the appropriate association of the other record.

As an illustrative example, consider a situation in which an author of municipal license record number 10098304 may desire to store this record in the database with a link to parent rabies vaccination certificate record number 100 76512. In this circumstance, the profile of the municipal license record may include one or more record properties that identify record number 10076512 as a parent document. The one or more record properties may also, if desired, identify the class of document to which the parent belongs. That is, the parent record may be classed as a rabies vaccination certificate record. In similar fashion, the profile of the rabies vaccination certificate record may include one or more record properties that identify record number 10098304 as a dependent record. The class to which the dependent document belongs, e.g., a municipal license record, may also be identified in the profile. Thus, entities that create records, especially entities that issue official license or certificate records, will easily be able to create and store information that links one or more records to one or more other records in the database.

Optionally, two or more records can be set up so that the term/expiration date of one record matches the tern/expiration date of one or more other records. For example, a municipal license record often is dependent upon a rabies vaccination certificate record and/or a rabies vaccination record. The municipal license record may be set up with an expiration date that matches the term of either one of its parent records. Such term matching may be accomplished by entering matching expiration dates manually with or without a prompt from the system. Alternatively, the system can be programmed so that matched terms for such documents are set up automatically upon creation of the records.

FIG. 6a shows an illustrative data entry form for entering data to create an owner information record. After being created, the record can be electronically stored. Data entry forms for entering other kinds of data by other kinds of offers would be similar except that the content fields would be different depending on the type of record and/or author of the record. In some embodiments, the record properties constituting the record profile may also differ, if desired. The author may be prompted to set appropriate record properties at a convenient time, e.g., when first saving the record. FIG. 6b shows a record property form by which an author can define the properties for a record.

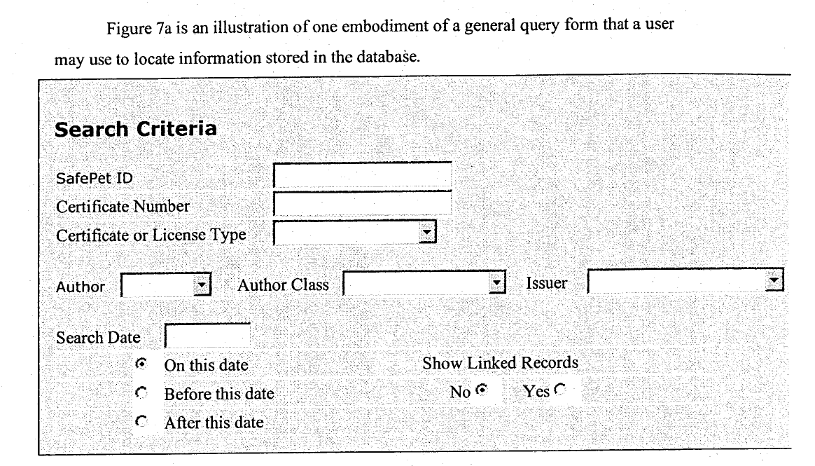

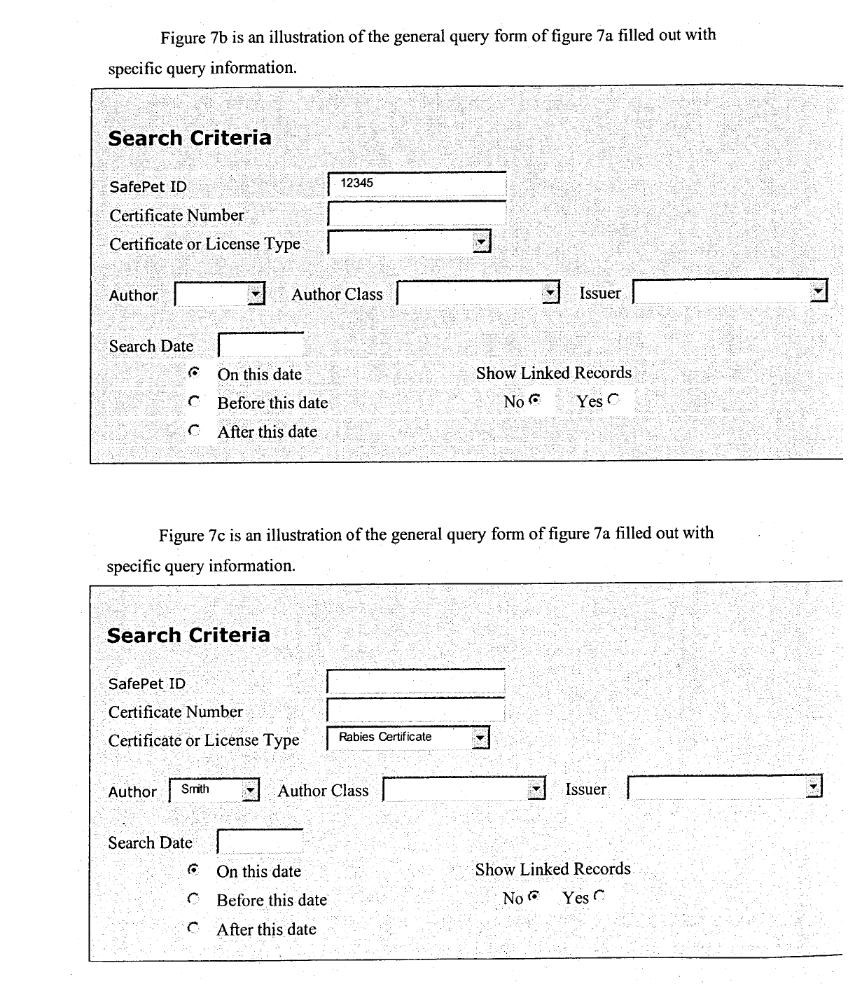

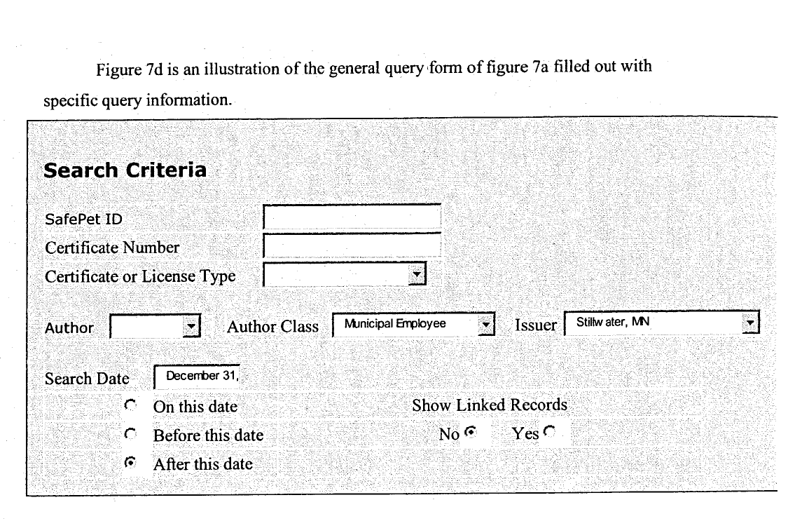

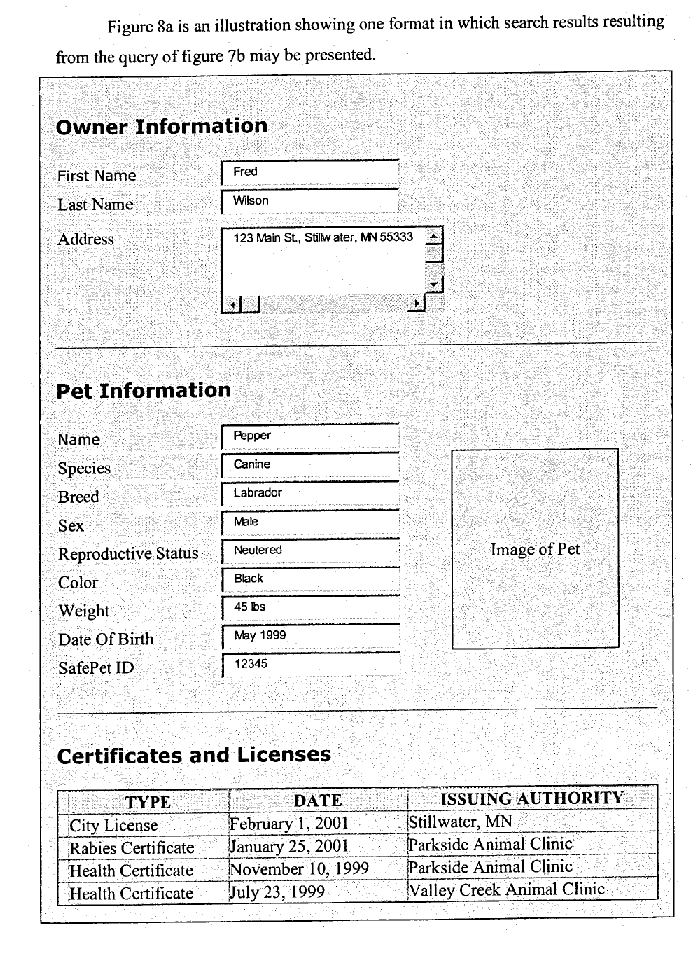

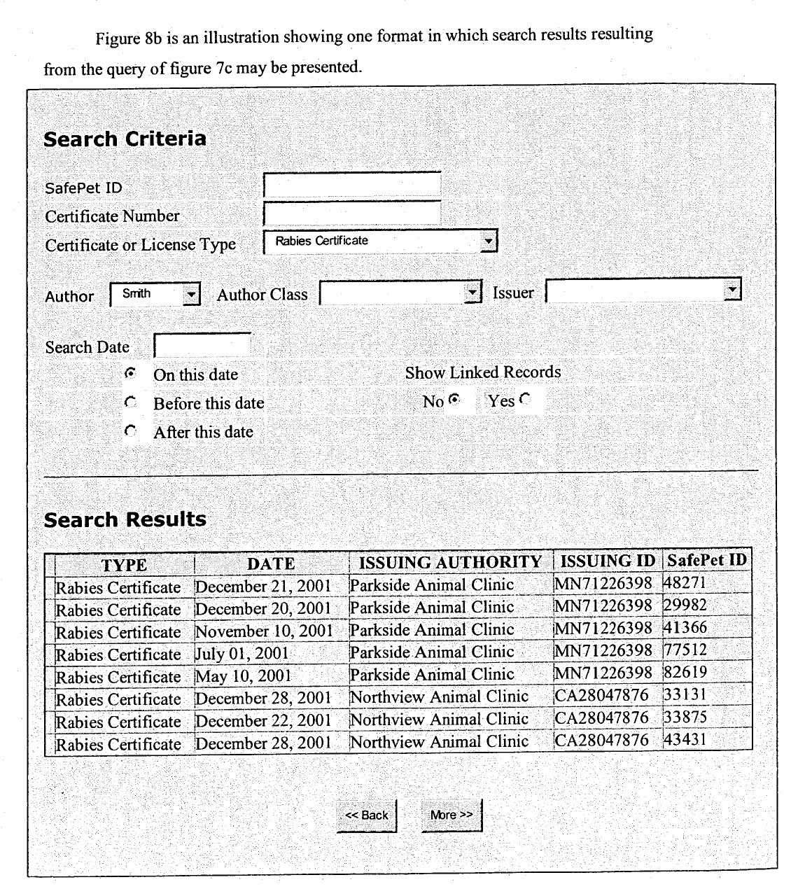

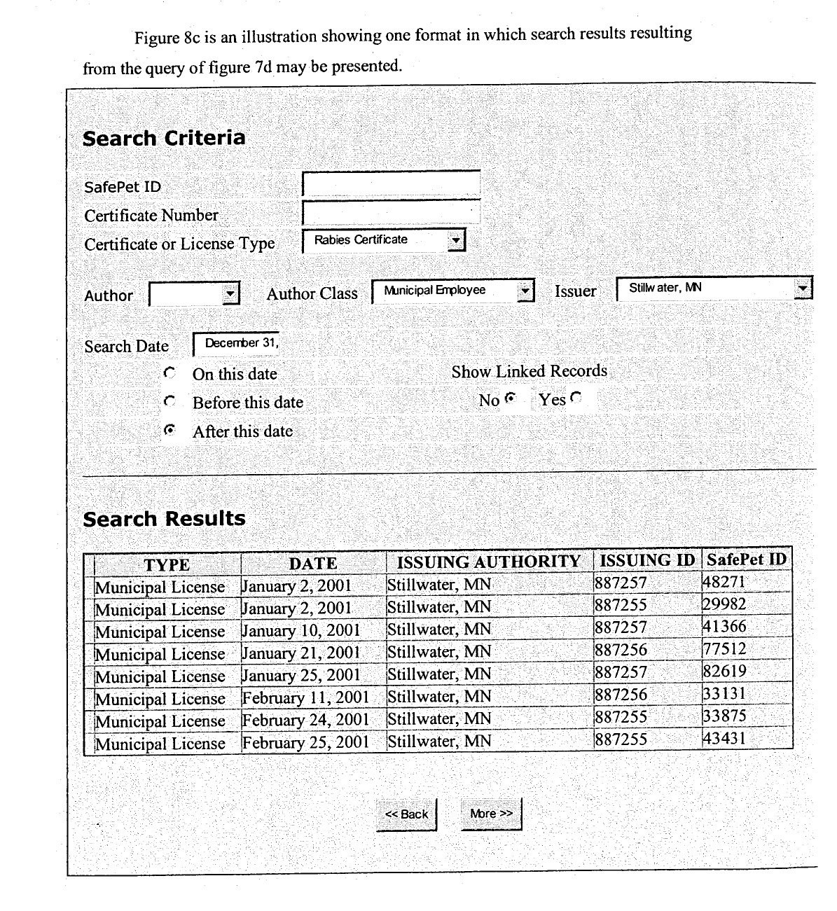

FIGS. 7a, 7b, 7c, and 7d show how document content and/or document properties may be used when querying the database to locate particular records. For example, FIG. 7a shows a blank query for locating records stored in the database. Selection criteria may be inserted into the query for any one or more document fields and/or properties in order to locate all records satisfying the selection criteria. For example, FIG. 7b shows a query in which the selection criterion constitutes an animal identification number. This query will locate all stored records corresponding to that animal identification number. Sample search results comprising a list of links to stored records satisfying the selection criterion are shown in FIG. 8a. FIG. 7c shows a query in which the selection criteria include the name of an author and the type of a certificate or license. This query will locate all stored records authored by the veterinarian Dr. Smith that are rabies certificate records. Sample search results comprising a list of links to stored records satisfying the selection criteria are shown in FIG. 8b. FIG. 7d shows a query in which the selection criteria include date information and a user class. This query will locate all stored records authored by Stillwater Municipal employees after Dec. 31, 2000. Sample search results comprising a list of links to stored records satisfying the selection criteria are shown in FIG. 8c.

Data stored electronically in the database of the present invention optionally also may be made available in one or more tangible counterparts. This option is preferred for data that may need to be presented to third parties from time to time. Such data includes, for example, official license data, official vaccination certificate data, health status data, and the like. Two preferred forms of tangible counterparts include tangible documentation and tangible fixtures.

Each of the tangible documentation and fixture embodiments independently comprises visually discernible and/or machine readable information that is a match for electronic data stored in the database. The tangible documentation embodiments may be carried by an animal owner or otherwise stored in a convenient location so that the tangible documentation is available on demand. The tangible fixture embodiments may be directly or indirectly attached to an animal. Both kinds of tangible counterparts have many uses. For example, the counterparts may be used by an owner to prove ownership. This can be important when a pet is lost or when a pet owner travels with a pet on a common carrier transport (e.g. airline). Depending upon the kind of information included in the counterparts, the counterparts can also be functionally equivalent to a vaccination certificate, an animal license, health status information, and/or the like. This kind of content is especially useful to have available when a pet owner interacts with third parties including airlines, groomers, kennels, pet show administrators, veterinarians, or other third parties who have a desire to know the legal or health status of an animal before rendering a service to the owner.

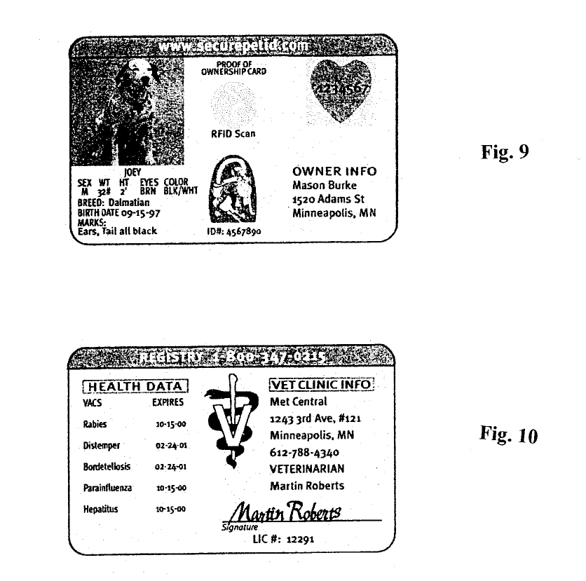

FIGS. 9 and 10 show a preferred embodiment of tangible documentation of the present invention in the form of a conveniently carried animal identification card. The card is wallet-sized and easily carried in a pocket, wallet, purse, or the like. The card is two-sided and carries useful information on both sides. First, the card may carry information describing one or more pet characteristics. Such information may include, for instance, a photograph of an animal in the same manner in which a driver's license includes a picture of the license holder. The card may also include additional pet information including the pet name, physical characteristics, birthdate, breed information, distinctive markings, and the like. The card may also carry owner information including, for instance, the owner name and address. The card may also include information about the current veterinarian clinic that provides health services to the animal. Such information may include the clinic name, address, phone number, veterinarian name, veterinarian signature, and veterinarian license number. Important health and vaccination information may also be included on the card. Such information may include important vaccinations and their expiration date.

The card may also include regulatory information such as official rabies and/or license information. For example, as shown in FIGS. 9 and 10, the card includes information corresponding to an official rabies vaccination certificate.

Three types of information preferably are included on the card to help link the card to the remote, centralized database in which information matching some or all of the card information is electronically stored. The first category of such information is the unique animal identification number which is analogous to a social security number. When communicating with the database, this number may be used to create, update, access, or otherwise use records corresponding to the animal. The second category of information is a physical description of the pet including a photo, breed, age, size, eye color and other distinguishing marks. The third category of such information includes information that helps a user contact the database. In one embodiment, this contact information is in the form of a web site address by which a user can contact the database over the Internet. Such information may also include a phone number by which a user may contact the registry by telephone, if desired. Advantageously, both types of contact information are included on the card to make contact with the database easier for persons having a computer with Internet access or a telephone.

Advantageously, the card may also carry other useful information, as desired. One example includes owner information as shown in FIG. 9. Official status such as rabies certificate information (shown in FIG. 10) may also be included. Other health-related information, e.g., vaccination status as shown in FIG. 10, of interest to third parties, e.g., airlines, kennels, groomers, etc., may also be included.

Preferably, the card is laminated to make it water-resistant, more durable, and/or more tamper-resistant. For purposes of authentication and/or tamper resistance, the card may optionally be encrypted with a code that has a unique association with the animal's ID number as associated in the central database. As one encryption option as shown in FIG. 9, an RFID chip storing a unique code may be incorporated into the card. As other options, magnetic encoding or any other technique that uniquely associates information on the card with information in a central database may also be used.

In practical effect, preferred embodiments of the card function at least in part has a health, proof of ownership, and/or rabies certificate that is provided in a tamper-resistant, durable, impermeable, easy to carry form. The card preferably complies with regulatory requirements to serve as an official rabies and/or health certificate or license. Encrypting with a code, e.g., via an RFID chip, provides the additional advantage of enhancing the tamper resistant and authenticity characteristics of the card. Preferred embodiments include at least four or more of the following elements:

1. rabies certificate information, e.g., DVM signature and other information required by applicable regulations and laws;

2. additional health information that might be useful to third parties;

3. owner information;

4. animal photo and other animal identification information;

5. laminated or other durable card format that is easily carried in a wallet;

6. unique code information to help maintain authenticity and enhance tamper-resistance; and

7. information associating the card with a remote system comprising a database and functionality to verify information on the card (Access to the system may be by automatic electronic authorization similar to that used for credit card authorization or other financial payment point-of-sale/service methods.

The card is easily carried on one's person and helps to establish the health status and/or ownership of the animal. The card may also acts as a pet passport when traveling. The contact information and animal identification code on the card also provides ready reference for contacting the database and creating, updating, accessing, or otherwise using records for the animal. Due to its simplicity, the card is readily re-issued with information about the animal changes. If a pet is injured while traveling, the veterinarian is easily contacted using the contact information if help to arrange emergency care for the pet is desired. The card preferably implements special printing and/or laminating techniques to help impede fraudulent tampering and to protect the card against water and other marring of the card material.

Tangible fixtures are items that may be directly or indirectly attached to an animal. Examples of tangible fixtures include tags, studs, stickers, tattoos, implants, embedded devices, clips, and the like. These fixtures may be used in addition to or as an alternative to tangible documentation counterparts of electronic data. Three kinds of tangible fixtures are most commonly used. The first of these is a rabies fixture (commonly known as a rabies tag), whose characteristics are described by the well-known Rabies Compendium. The kind of information that may be placed onto a rabies fixture depends on local laws and regulations, but typically includes one or more of a rabies certificate or tag number, the year in which the vaccination is current, veterinarian clinic name, city, state, and indicia that identifies the fixture as relating to a rabies vaccination. Official regulations typically specify the shape, color, thickness, and/or content of these fixtures.

A second, commonly used tangible fixture that may be attached to an animal is an animal license fixture, e.g., a municipal or city license. As is the case with a rabies fixture, the contents included on a license will depend upon local laws and regulations. Typically, information included on a license fixture includes one or more of the year in which the license is current, a license number, city, state, and the contact information such as a telephone number for the licensing authority.

A third, commonly used tangible fixture that may be attached to an animal is a fixture including owner/custodian identification information and may exist in a variety of forms. Typical information on one of these includes a pet name, owner name, and owner contact information.

In addition to the substantive information included on each of the three types of tangible fixtures noted above, one or more of these may also include one or more of the unique animal identification code and the database contact information as noted above with respect to the tangible documentation. The unique code is especially useful to help match the animal and/or information on the fixture with corresponding electronic counterparts in the database and/or information for contacting the database in which counterpart electronic records for the animal are stored. As is the case with the tangible documentation, such information may be incorporated in machine and/or human readable formats. It is preferred that at least human readable embodiments of such information are included so that the unique identification code and database contact information are readily viewable by an entity who interacts with the animal.

Particularly preferred embodiments of tangible fixtures are described in U.S. Pat. No. 6,283,065, incorporated herein by reference. Generally, this patent describes petwear in the form of pet identification necklaces and containment collars in which collar studs or other collar mounted accessories are used in the place of dangling tags to display information. In preferred embodiments, information is applied onto such studs or accessories in the form of stickers that are easily updated by applying new stickers over the old or by removing the old sticker before the new one is applied. The stickers may be substitutes for any conventional tags, including owner tags, municipal license tags, or vaccination certificate tags.

Both the tangible documentation and fixture embodiments include features that help to make it difficult to copy, counterfeit, or otherwise tamper with the embodiments. Specifically, both may include a unique, machine readable, encrypted code, which may be the same or different from the unique animal identification code. This machine readable code may be encrypted in a magnetic recording strip, bar code, RFID chip, or the like. As shown in FIGS. 9 and 10, the machine readable code is embodied in an RFID chip. An identical code will be electronically stored in the database. The encrypted code and its counterpart in the database must match in order for the tangible counterpart to be authentic.

Advantageously, the infrastructure to implement such verification procedures is readily available. The magnetic and bar code verification techniques that may be incorporated into the card would use equipment and system infrastructure that already serves the electronic financial payments (e.g. credit card) and product identification industries. The RFID chip option uses an equipment infrastructure that has been set up in the pet industry.

As additional security, other information on a tangible embodiment also must match corresponding data electronically stored in the database in order for the embodiment to be authenticated. Thus, to determine the authenticity and veracity of information on the embodiment, a user simply accesses the database and uses the unique animal identification number on the tangible counterpart to access electronic records for the animal. If the information stored in the database matches information on the counterpart, the counterpart may be deemed to be authentic, and the veracity of information appearing on the counterpart is confirmed. The system thus provides a method enabling third parties requiring such information to easily and conveniently verify tangible information via the centralized database. Alternatively, independently of any tangible information, an entity may elect to query the database using electronic online methods to determine animal information.

As one example of this kind of verification conduct, an airline may require proof of a rabies vaccination certificate and/or other health status before allowing a pet to travel on the airline. In addition, the airline may wish to verify the identity of the pet to make sure that the online information does in fact correspond to the animal seeking transport. In some cases, the pet owner or custodian may present the airline with tangible documents describing the animal's status. In other cases, the owner may not have any tangible documents on hand that may be requested by the airline. In either case, the airline can access the database, locate the pertinent records for the animal, and confirm both the identity of the animal and the animal status. If the online information matches the information presented to the airline, travel may be allowed. On the other hand, if there is a discrepancy between the online information in the information presented to the airline (e.g., the physical characteristics of the animal seeking travel do not match the online information), the airline may refuse travel or otherwise follow-up to determine why there is a discrepancy.

As another example of such verification conduct, any authority that requires proof from a pet owner (custodian) of a pet's vaccination status can use the card form presented by the owner. The card can also serve as an identity card for the pet based on a photo and physical description as well as a proof of ownership by the pet owner. An “inspecting” or “inquiring” authority can verify card information by contacting the database. Similarly to other electronic verification systems in use today for financial payments (credit card, check verification, etc.), verification can be electronic or operator assisted.

It can be appreciated that the system of the present invention functions optimally when different classes of users interact with the system. In particular, veterinarians and municipal authorities are two important classes of users who provide a foundation for the system as a whole. Many key records contained in the system generally may only be created under the direct or indirect authority of one of these entities. Additionally, although one objective of the system is to serve as an electronic repository of important animal information and documentation, the system is also intended to be a resource for pet owners, “good Samaritans”, and a variety of different inquiring third parties. Thus, other users are important as well.

Accordingly, the system of the present invention provides multiple avenues by which animals and different classes of users may be registered with the system. Such avenues include the internet, telephone, facsimile, mail, registration service, and the like. Generally, a user registers with the system in order to gain access whose scope depends upon appropriate factors including the class to which the user belongs. If the user indicates during the course of registration that ongoing interaction with the system is contemplated, the system will assign login credentials to the user. Login credentials preferably include a username and password.

Preferably, users may select from a menu of subscription plans when registering to have access to the system. The system preferably will automatically track subscriptions to assure that a putative user attempting to log in is authorized to access the system. Invoices or confirmations of payment for subscription services may be automatically created and submitted to the subscriber using any combination of point-of-sale, electronic commerce and/or traditional mail delivery. Similarly, remittance of an invoice may be performed using point-of-sale payment, c-commerce methods and/or traditional mail. In addition, subscription services involving linked records can be issued, invoiced and payment remitted through a consolidated process. For example, veterinarian clinics providing rabies vaccination and/or spaying or neutering of pets, if designated to be an “agent” for a municipal licensing agency, can create and issue a dependent, official record such as a pet municipal license as part of a single consolidated transaction.

The first step for a pet owner to subscribe to the service is to purchase a tangible fixture upon which a unique animal identification code is printed. This tangible fixture is attached to the pet providing a secure form of visual identification of the unique identification code and directions for accessing the on-line pet database for the purposes of return of a lost pet or verification of pet health, license and/or certificate status.

The second step in the subscription process is to register the pet and owner contact information on the central verification database. The registrant may be prompted to input some mandatory and some optional information about the animal To help ensure that the registrant remembers the code to facilitate future communications about the animal, the system may provide the registrant with one or more tangible and/or electronic copies of items comprising the code. For example, an e-mail including the code could be sent to the registrant. Tangible documentation may also be created and provided to the registrant. The system may also include on-line methods to obtain the code in the event that indicia of the code are lost or not on hand.

Advantageously, the system does not rely solely upon a pet owner to help ensure that animals are registered with the system and their records properly entered and maintained. Importantly, the veterinary clinic is important in this regard because it offers an opportunity to link the provision of veterinary services with system registration. That is, the veterinarian can be an important catalyst to initiate and perfect animal registration. This role is well-suited to the veterinarian inasmuch as many kinds of records desirably stored in the database are created by the veterinarian or are dependent upon records created by the veterinarian. In connection with rendering health services, the veterinarian can easily access the database, register an animal if needed, update health and vaccination records, issue official documents in both electronic and tangible form, and the like.

Another major collaborative institutional entity group is municipal government through its licensing and control of pets as part of local government's public health responsibilities to control over-population of pets. The system advantageously provides the opportunity to consolidate all identification tagging systems (rabies, city license and pet identification) into a single, more streamlined system that serves all functions more effectively. The system is designed to facilitate collaboration with municipal governments in order to provide pet licensing.

Additionally, the system provides an opportunity to compile historical pet healthcare and mortality information for use by various non-profit and for-profit entities conducting research on pet epidemiological, loss-control and, perhaps, other heath care related subjects. These entities may be provided database access for research purposes insofar as the research does not invade the privacy of pet owners.

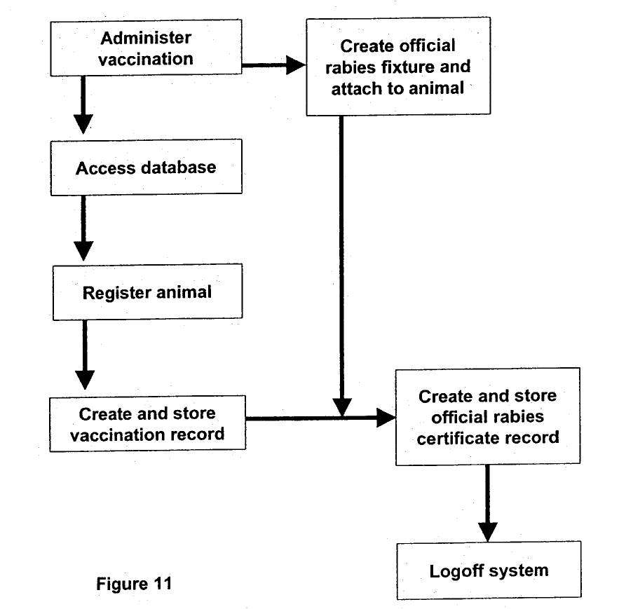

FIG. 11 schematically shows one method by which the system of the present invention can be used in connection with an animal vaccination of an animal that has not yet been registered in the database. In a first step, a veterinarian administers a vaccination to the animal. After the vaccination is administered, the veterinarian, or someone under the authority of the veterinarian, accesses the database of the present invention remotely through a network, Internet, or other suitable interface. The user will also create and store a vaccination record in the database that comprises information relating to the vaccination that was just administered to the animal. A licensed veterinarian professional under whose auspices the vaccination was applied shall certify that such vaccination information is true and accurate. The user typically will also enter information about the animal in the database, such as pet characteristic information, owner information, veterinarian clinic information, health-related information and/or the like. If authorized by the appropriate regulatory authority, the user may also create and store an official, electronic rabies certificate record that is dependent from the rabies vaccination record. In the meantime, the user may create or otherwise provide a tangible, rabies element (e.g., a tag, stud, sticker, or the like) and attach it directly or indirectly to the animal. The tangible rabies element generally comprises not just human and/or machine readable information documenting the rabies vaccination in some fashion, but preferably also a unique code (which may be the same or different than the unique animal identification code) that provides a link between the user of the database and one or more electronic records stored in the database and relating to the animal. The human and/or machine-readable information may also include information that helps a user contact and access the database. Such information could include, e.g., a phone number, mailing address, web site address, or the like.

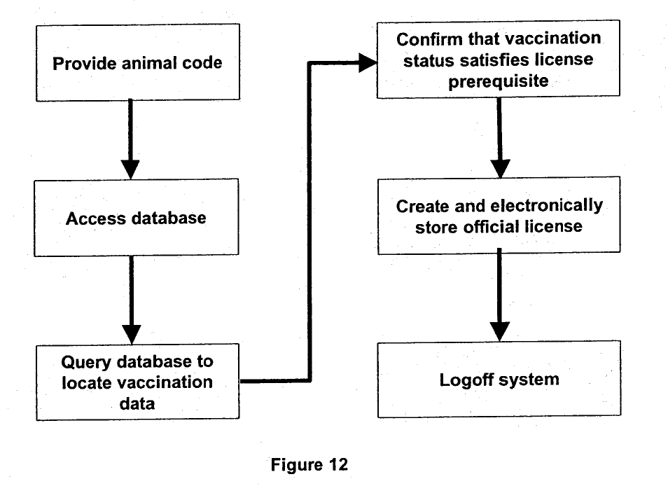

FIG. 12 schematically shows one method by which the system of the present invention can be used to issue an official animal license for an animal wherein the license is dependent upon one or more vaccination records already stored in the database. First, a user determines or is otherwise provided with information that provides a link to the electronic records stored in the database relating to the animal. Such information generally will comprise a unique code associated with the animal. The user accesses the database and queries the database to locate one or more electronic rabies vaccination records, if any, for the animal. One or more rabies vaccination records or absence thereof will indicate the vaccination status of the animal. From this information, the user will be able to determine whether the vaccination status of the animal satisfies the prerequisites for the official animal license. If the prerequisites are satisfied, the user can create and electronically store an official license record in the database. Optionally, the user may cause a tangible license element (e.g., a tag, stud, sticker, or the like) to be attached directly or indirectly to the animal. In a fashion similar to the rabies element discussed above, the license element may include human and/or machine-readable information documenting the license in some fashion, linking information, and database contact information.

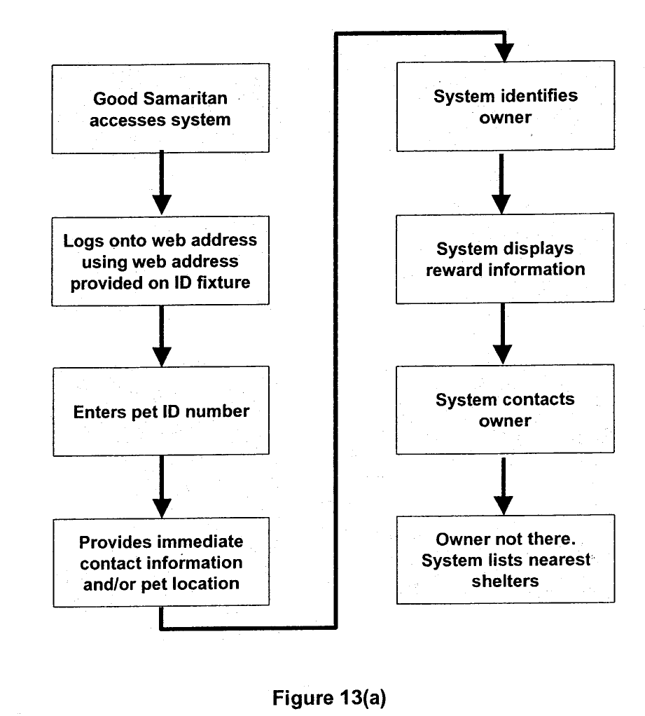

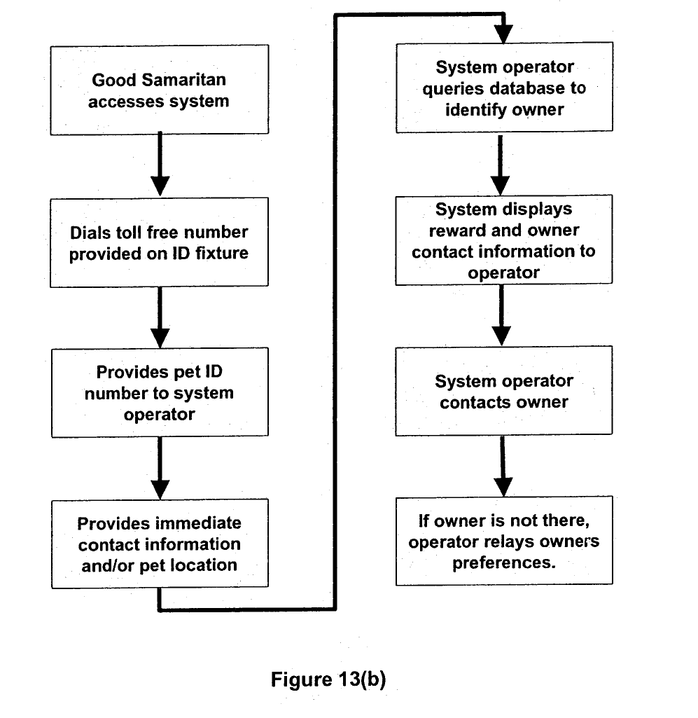

The system of the present convention can be used to return a lost pet to its home when the lost pet is wearing a tangible fixture. The tangible fixture includes two important pieces of information that are especially useful for a “good Samaritan” who finds the lost pet and desires to help return it to its home. FIG. 13a schematically shows one method by which this may be accomplished in the practice of the present invention. Using the information on the tangible fixture, the “good Samaritan” can contact the database, enter the identification code that identifies the animal, inform the database that the animal is lost, and provide the database with information explaining how the owner of the animal may reach the “good Samaritan” to recover the animal. The system will then use such information to determine the identity of the owner. Optionally, after the “good Samaritan” has inputted the lost pet information into the database, the system may automatically determine whether the owner has offered a reward for anyone who finds lost pet and can then display reward information to the “good Samaritan”. Once the owner is identified, the system automatically generates a communication that informs the owner that the lost pet has been found and how the lost pet may be recovered. The owner may communicate with a “good Samaritan” directly or through the system in order to recover the lost pet. Advantageously, the system serves as an intermediary between the “good Samaritan” and/or the pet owner so that the privacy of the pet owner and/or the “good Samaritan” is not compromised. Advantageously, the unique animal identification code worn by the animal thus allows a user such as a “good Samaritan” to easily communicate with the database about an animal for which nothing is known except for the unique code.

FIG. 13b schematically shows a second optional method by which a “good Samaritan” can use the information on the tangible fixture to contact a pet owner and arrange for return of the lost pet to its owner. The “good Samaritan” can use the toll free telephone number displayed on the fixed fixture to call a 24 hour a day operator who accesses the database of the present invention and provides appropriate information to enable return of the pet to its owner.

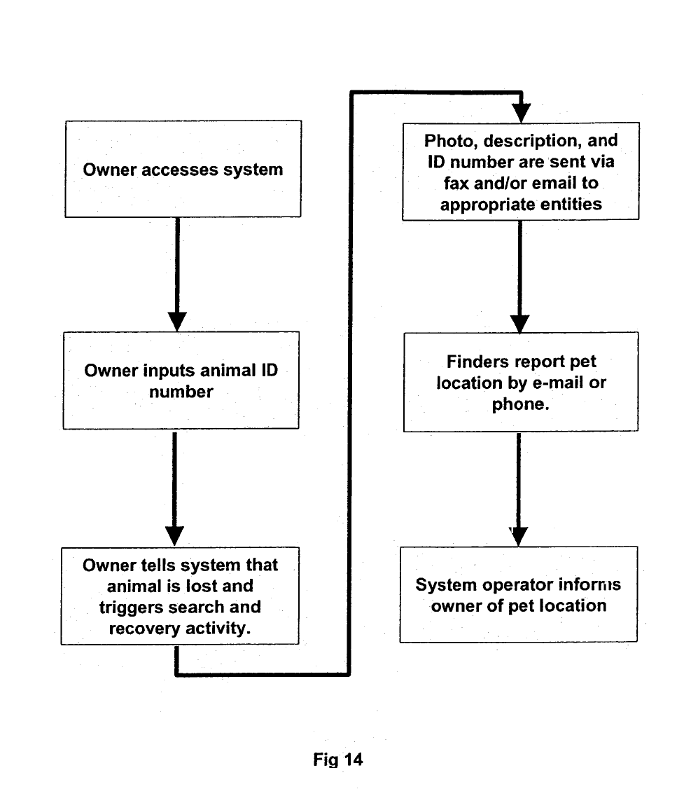

FIG. 14 schematically shows a method of the preferred system in which the owner uses the system of the present invention to initiate specific and proactive search and recovery activity. The pet owner can log on to the search and recovery component using their owner name and password and then access information pertaining to their lost pet. After entering information describing the location where the pet was last seen and other pertinent information relating to the incident the owner can elect to send pet identification and owner contact information to agencies, shelters, clinics and other entities who have agreed to participate in the search and recovery process. Information may be sent electronically via fax or email to fax numbers and email addresses for the recipient search and recovery entities stored in the search and recovery database. The owner may also retrieve telephone numbers, address and other pertinent information pertaining to the search and recovery entities that will allow the owner to contact such entities directly. The owner may also print posters with picture and other pet identification information and owner contact information that can be posted by the owner in locations that might be beneficial to the recovery of the lost pet.

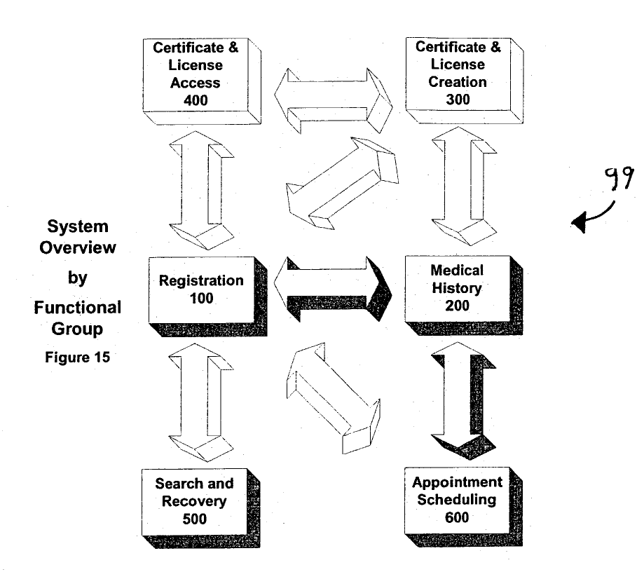

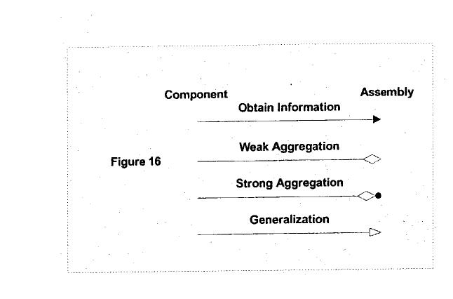

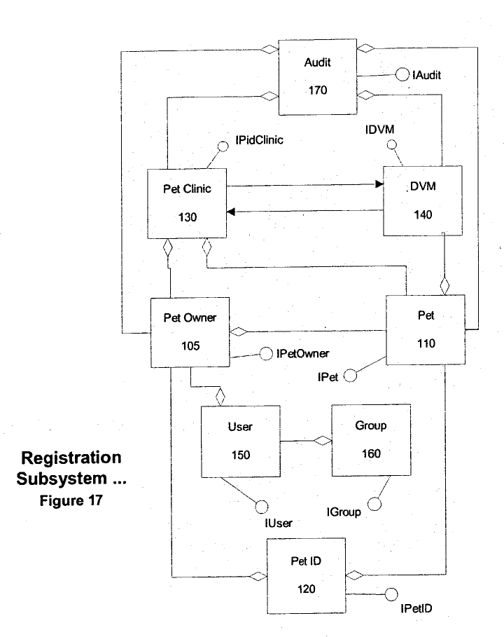

FIGS. 15 to 23 show one preferred embodiment of a system architecture 99 for implementing the principles of the present invention. The preferred system architecture 99 includes six subsystems that interact with each other to perform the desired functions. These functions include: (1) Registration subsystem 100; (2) Medical History subsystem 200; (3) Certificate and License Creation subsystem 300; (4) Certificate and License Access subsystem 400; (5) Search and Recovery subsystem 500; and (6) Appointment Scheduling subsystem 600. In FIG. 15, the boxes represent the particular subsystems and the arrows indicate preferred subsystem interactions. The functions of each of these subsystems preferably are implemented by a number of software components. Any particular software component may be used to perform functions in one or more of the subsystems. Preferred software components used by each subsystem are shown in FIGS. 17 to 23. These Figures use a number of different arrow types to schematically represent the interaction among the various software components. For reference, FIG. 16 defines these arrow types. Generally, the arrows define the direction of aggregation from lower level components or elements into higher level assemblies.

As an overview of system 99, the Registration subsystem 100 is the hub of the system 99 insofar as the system 99 revolves around the registration of entities related to an animal, e.g., animal, livestock, or the like, and the animal's unique system identification as well as the various entities that may have a need to submit or retrieve data pertaining to the animal. Security functions and controlling access rights to the data are also performed by one or more components of the Registration subsystem 100.

The functions of registration subsystem 100 are used when a veterinary clinic registers itself with system 99. During the registration process, pertinent information pertaining to the clinic is entered though a suitable user interface and is stored in a system database. Such information may include clinic name, address, contact information, DVMs providing services at the clinic; other clinic service providers; clinic and service provider scheduling availabilities and protocols; clinic procedure information and the like. As another example, animals may be registered with system 99 through a variety of interfaces, including phone, e-mail, internet, regular mail, facsimile, or the like. Registration may be performed by a variety of individuals, including one or more of an animal owner, a vendor employee, a municipal official, or the like.

Users of the system are assigned a login user ID by registration subsystem 100 after completing the registration process. Once a user is assigned a login ID, the user will be assigned a role within the system, and this role is at least one factor in determining the user rights that the user will have for security purposes. Consequently, it is preferred that every user of the system will be required to log into the system using their unique user identifier and a password. As an option, third party “good Samaritans” who recover missing animals and report this to system 99 may or may not be required to use a login ID or password in order to use the system for animal recovery purposes.

A user may have multiple roles. Under certain circumstances a role may be automatically assigned to a user based upon information provided during registration. In other circumstances, a role may be manually assigned, e.g., vet clinic administrator or the like. Examples of roles that users may have within system 99 include animal owner, municipal employee, airline employee, groomer, DVM, combinations of these, and the like. The Registration system 100 preferably will track whether a user is authorized, either as an employee or agent, by a regulatory entity (e.g., state, administrative agency, municipality or other governmental body) to use system 99 to input, create, modify, update, store, or otherwise manipulate data relating to official documents, e.g., official licenses or certificates.

The various system interfaces will use the user group privileges to which the user belongs, as one way to control access to data in system 99. Additionally, system 99 may comprise a number of user interfaces that are presented to a user depending the type of user group to which a user belongs. The system components responsible for providing a user interface to a user preferably may be either stand-alone applications or web server-based applications. Because a particular interface may be customized for a specific class of user, queries made to the database can be can be limited to retrieve only those records needed to support the given class.

A preferred embodiment of software groups and components that interact to perform the functions of registration subsystem 100 is shown in FIG. 17. Constituents including three subgroups of software components perform the functions of the preferred registration subsystem 100. The core animal and owner ID subgroup 103 includes the animal owner software component 105, the animal software component 110, and the animal ID software component 120. The three components in this subgroup manage all of the core identification data regarding the owner, the animal and the linking animal ID number. The veterinarian clinic registration subgroup 107 includes the animal clinic software component 130 and the DVM software component 140. The two components included in this subgroup extend the registration function beyond the owner and animal to include veterinarian service providers. The security subgroup 109 (also referred to herein as “security components”) includes the user software component 150, the group software component 160, and the audit software component 170. Security functions, e.g., access to the registration components as well as other subsystem components, is controlled by these security components within the registration subsystem 100.

Medical History Subsystem 200

The Medical History subsystem 200 controls the collection, use, modification, updating, and integrity of medical data for animals such as animals, livestock, and the like. As noted below, this medical data may be used to help generate and issue electronic and/or tangible embodiments of official data (medical certificates, licenses, etc.) created by a qualified medical professional, regulatory entity, or other authority. For example, if the type of procedure is a vaccination, then the corresponding data may be used to create a corresponding official vaccination certificate or electronic counterpart thereof. Data for some procedures that are not vaccinations also may be used to generate official certificates. Examples of such procedures include spaying, neutering, and de-worming, or essentially anything that might be used by a kennel, veterinarian, government agency, or other authority to determine the health or reproduction disposition of an animal.

When an animal is treated at a clinic or other vendor such as a groomer or the like, the Medical History subsystem 200 is used to input, create, modify, update, store, or otherwise manipulate data relating to the treatment in medical history records of a system database. Data relating to all types of procedures performed at a clinic or by another animal care vendor may be stored in a system database. As an animal is treated, for instance, the type of procedure performed on the animal, along with date, time, attending DVM or service provider, clinic or other entity, animal identification data, animal owner information, etc. may be stored as a medical history record.

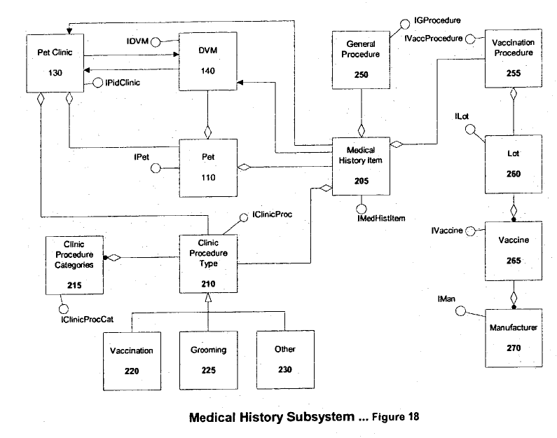

A preferred embodiment of software components that interact to perform the functions of a preferred Medical history subsystem 200 is shown in FIG. 18. These software components include animal component 110, animal clinic component 130, DVM component 140, medical history item component 205, clinic procedure type component 210, clinic procedure categories component 215, vaccination component 220, grooming component 225, other component 230, general procedure component 250, vaccination procedure component 255, lot component 260, vaccine component 265, and manufacturer component 270.

### Certificate and License Creation Subsystem 300

The certificate and license creation subsystem 300 includes functions to input, create, modify, update, store, or otherwise manipulate data relating to the creation of electronic and/or tangible, official certificates and licenses. The integrity and veracity of official data/documents is important. Accordingly, subsystem 300 uses security components to help assure that official data/documents, whether in electronic or tangible form, can only be generated, accessed, or otherwise used by an authorized entity in a manner consistent with the security rights allocated to such entity. Preferably, certificates may only be generated for animals that are registered with the system 99.

It is common for a certificate, e.g., a vaccination certificate, to be created as a consequence of a medical treatment, e.g., a vaccination. That is, after a qualifying clinic medical procedure has been administered to an animal, an electronic certificate may be created based, at least in part upon that treatment. Accordingly, subsystem 300 includes functionality to help accomplish this.

For example, to generate an official certificate, a DVM that administers a qualifying medical procedure uses a proper user ID to log into the system 99, e.g., via the clinic PC on which an appropriate software interface resides or is accessed. The software interface will guide the DVM through a process that involves identifying the registered animal that received the treatment and recording the medical procedure information for the procedure performed on that animal. Each medical procedure performed on an animal at a clinic can be recorded in the system, but not all procedures will qualify for electronic certificate creation. Typically, under local laws and regulations of many jurisdictions, vaccinations and spay/neuter operations are procedures that provide a basis for a certificate to be generated. Additionally, in some localities, treatments for parasites such as ticks, worms and fleas may also trigger an event whereby a certificate may be generated.

When the qualifying medical procedure has been entered into the system and assigned to a registered animal, the software preferably will prompt the DVM to determine if a corresponding official certificate is to be generated. If the DVM indicates that a certificate is to be generated, the subsystem 300 prompts the DVM to input any additional information that might be needed but otherwise automatically generates an electronic and/or tangible counterpart of an official certificate. Information incorporated into the certificate is electronically archived and retrievable on demand so that a subsequent change to any of the underlying information will not invalidate the certificate, change its content (if no change is desired), or compromise its veracity. For every certificate that is created, an audit record preferably is created and saved in system 99. Thus, certificate content will be verifiable at least over the life of the certificate. As data for certificates are stored, the data preferably includes a certificate expiration date and a unique identification number. The unique identification number can be used to directly and easily access the certificate on demand.

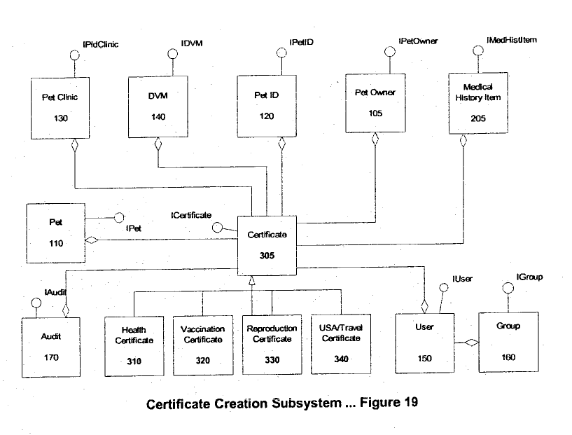

A preferred embodiment of software components that interact to perform the functions of creating a certificate via the certificate and license creation subsystem 300 is shown in FIG. 19. These components include animal owner component 105, animal component 110, animal ID component 120, animal clinic component 130, DVM component 140, user component 150, group component 160, audit component 170, medical history item component 205, certificate component 305, health certificate component 310, vaccination certificate component 320, reproduction certificate component 330, and USA/Travel certificate component 340.

In contrast to a certificate which typically may be generated based upon medical information, license creation and issuance typically requires that some underlying certificate exists. In the case of a municipal dog license, for example, a vaccination certificate and perhaps a health certificate indicating the animal's reproduction status are often both prerequisites for a license. Consequently, license creation also is a function of the Certificate subsystem 300. Creating a license not only involves actions including verifying that the underlying certificate exists and is valid, but it also involves collecting owner, animal and ID information pertinent to the license. A copy of the gathered information will be archived so that if any of the underlying information changes after the license is issued, the license information will remain in the system database as it existed at the time of licensing. License creation events preferably are captured in the system audit file as well. Only authorized users may create, access, or otherwise use licenses and data relating thereto.

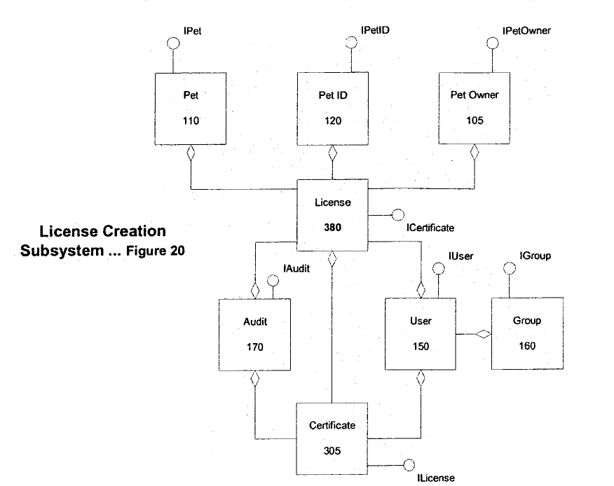

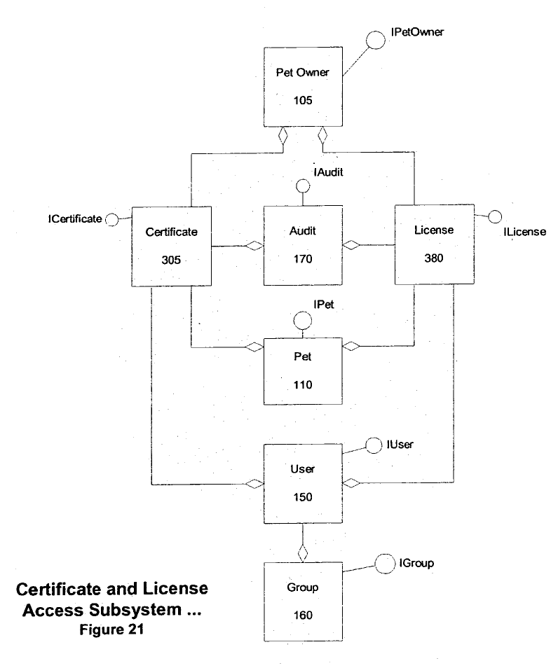

A preferred embodiment of software components that interact to perform the functions of creating licenses in the certificate and license creation subsystem 300 is shown in FIG. 20. These components include animal owner component 105, animal component 110, animal ID component 120, user component 150, group component 160, audit component 170, certificate component 305, and license component 380.

### Certificate and License Access Subsystem 400

Authorized users of the system may access certificate and/or license information maintained in a system database. The security components will allow or deny users access to certificate and/or license information based on information comprising user ID, user group, and/or the like. For instance, DVMs, regulatory authorities, public transportation authorities, groomers, kennel operators, border control officers, animal control officers, and the like may be able to view certificate information of multiple animals in the system database, as can municipal licensing center users, kennel operators, etc. On the other hand, individual animal owners may be allowed access to certificate and/or license information related only to the animals that they own and to the animals for which they currently act as an agent. In some instances, to maintain privacy, no other information pertaining to the DVM, animal, animal owner, or clinic preferably will be displayed to the user when viewing certificate and/or license data.

Certificate and license information may be retrieved using appropriate data stored in system 99, including one or more of the document number, the animal ID, animal owner, DVM and/or DVM clinic, breed, date of creation, date of expiration, document type, document author, document edit date, or any other type of data relating to the document. Preferably, only the current certificates of a given animal may be retrieved and viewed through any of the given interfaces, and expired certificates will not be accessible to a typical user. Preferably, every time a certificate is accessed within the system, an audit record is saved which may include data indicating one or more of which certificate was viewed, by whom, from where it was viewed, and when it was viewed.

A preferred embodiment of software components that interact to perform the functions of certificate and license access subsystem is shown in FIG. 21. These components include animal owner component 105, animal component 110, user component 150, group component 160, audit component 170, certificate component 300, and license component 380.

### Search and Recovery Subsystem 500

The Search and Recovery subsystem 500 performs functions to help locate the owner of a found animal or proactively to launch search and recovery activities on an animal that has been reported as lost. For example, when an animal owner discovers that an animal is missing, the owner may log onto the system and input information relating to the missing animal. Preferably, the subsystem 500 then automatically notifies regional shelters, kennels, police, other animal owners, veterinarian clinics, system operators, and/or the like via a suitable communication, e.g., an e-mail, that an animal has been reported missing. The notification may contain missing animal information, preferably including a missing animal poster or similar image. Activities pertaining to the search of the missing animal may be recorded in the animal's missing animal message board thread. All or only portions of the animal's message board thread may be viewed by anyone. When the animal is found, an entry is made in the missing animal message board thread indicating that the animal has been recovered. The finder, system operator, or automated functionality may then make arrangements for the return of the animal to its owner. It is anticipated that animals may be recovered before they are even discovered or reported by the owner to be lost. When an animal is found that has not yet been reported missing, a missing animal message board thread is created (by the finder or system operator) for that animal and the owner (or designated contact) is notified, e.g., via e-mail and/or telephone calls.

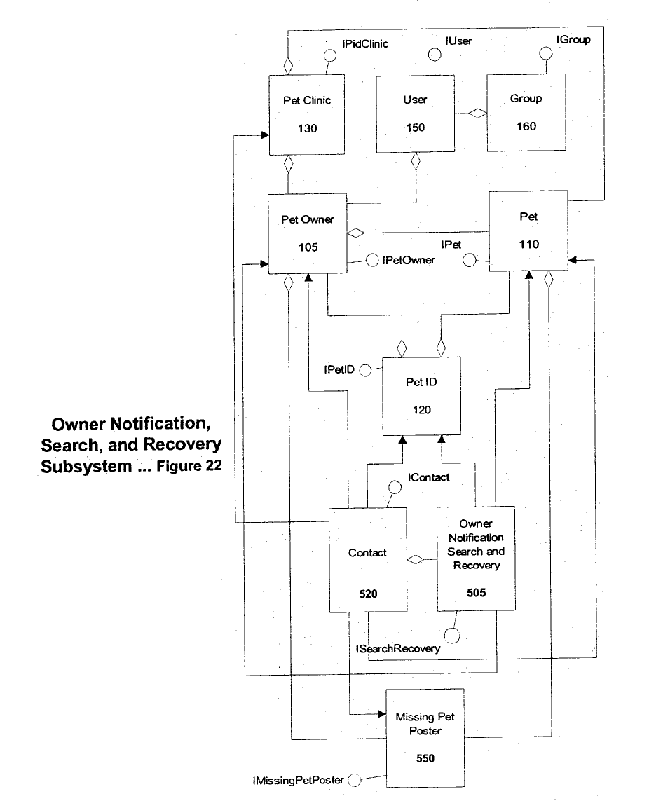

A preferred embodiment of software components that interact to perform the functions of the Search and Recovery subsystem 500 is shown in FIG. 22. These components include animal owner component 105, animal component 110, animal ID component 120, animal clinic component 130, user component 150, group component 160, owner notification search and recovery component 505, contact component 520, and missing animal poster component 550.

### Appointment Scheduling Subsystem 600

The Appointment Scheduling subsystem 600 provides scheduling services to allow animal owners to schedule appointments at veterinary clinics or other participating vendors for their animals. For example, when a user desires to schedule an appointment, the vendor's current schedule may be checked for availability. Preferably, the schedule of each person that may provide a service must be accessed to ensure that over-allocation of resources does not occur. For each scheduled appointment, an appointment type may be specified. The system may also automatically determine the length of the appointment based on information including the appointment type and/or the type of animal. Because service providers may act in more than one role, e.g. a DVM may also act as a groomer, the role the service provider will play is stored along with the other appointment information.

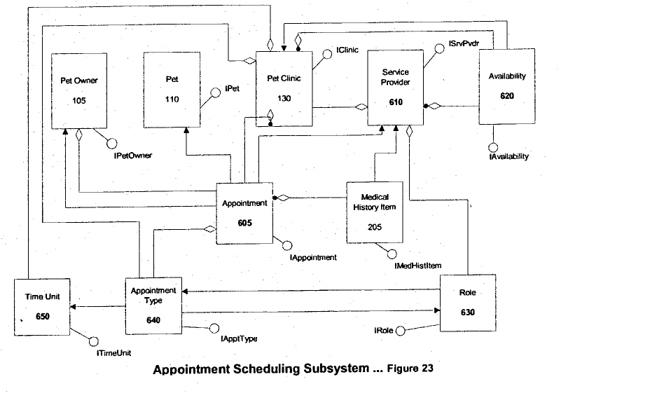

A preferred embodiment of software components that interact to perform the functions of the Appointment Scheduling subsystem 600 is shown in FIG. 23. These components include animal owner component 105, animal component 110, animal clinic component 130, medical history item component 205, appointment component 605, service provider component 610, availability component 620, role component 630, appointment type component 640, and time unit component 650.

The preferred software components as shown in FIGS. 17 to 23 will now be described in more detail:

### Animal Owner Component 105

The animal Owner Component 105 defines the system's concept of an animal owner. An animal owner's properties, methods and access are controlled through the animal Owner Component 105. Properties of an animal owner include data items that describe an animal owner or are used to assist the application in processing animal owner information. Examples of an animal owner's properties include: animal owner name, animal owner address, animal owner telephone number, and animal owner status.

An animal owner method is software that manipulates Animal owner properties and manages the creation, deletion or updating of system Animal owner components stored either in memory, the database or both. Access to Animal owner properties and methods are performed in conjunction with the security components.

The animal Owner Component 105 also is used at least in the following subsystems: the registration, search and recovery, certificate and license creation, certificate and license access, medical history, and appointment scheduling. The animal Owner Component 105 is used during the owner and animal registration process. During this process information pertaining to the owner of the animal being registered is obtained from the user interface and stored in the system database by the animal Owner Component 105. Associations to clinics, animals, DVMs, IDs, and related entities are made preferably at this time. If a currently registered owner registers a new animal, the new animal is linked with the existing owner information.

At the time of registration, the animal owner Component 105 interacts with the animal Component 110 to obtain a reference for association purposes to the animal being registered. The animal Owner Component 105 interacts with the animal ID Component 120 to supply a reference to itself so that an animal ID may be used to find the associated animal owner or animal quickly. The animal Owner Component 105 interacts with the animal Clinic Component 130 to supply a reference to itself for association purposes. The animal Owner Component 105 interacts with the User Component 150 to supply information to be stored with the user account record. Finally, the animal owner component interacts with the Audit Component 170 to log changes in the animal owner component data.

In the Search and Recovery subsystem 500 of FIG. 22, the animal Owner Component 105 is used to obtain animal owner information to create a missing animal message board thread, a missing animal poster, and gather owner and owner agent contact information. The animal Owner Component 105 interacts with the owner Notification, Search, and Recovery Component to provide information about the owner of the lost animal that is to be used to create the missing animal message board thread. The owner Notification, Search and Recovery Component 505 interacts with the Contact Component 520 to provide information used to notify an animal owner about a missing animal. It interacts with the Missing Animal Poster 550 to provide information about the owner of the missing animal that will be used in the creation of a missing animal poster.

The animal Owner Component 105 interacts with the User Component 150 to obtain access permissions required to begin the process of reporting a missing animal. The animal owner component may interact with the animal ID Component 120 to supply a reference to itself to gather information about the animal owner that will subsequently be used to report a missing animal.

The Certificate Create and Medical History subsystems 300 and 200 use the animal Owner Component 105 to obtain owner information that is to be stored with the certificates and licenses issued for an animal. The animal Owner Component 105 interacts with the Certificate Component 305 to provide information about the animal owner for inclusion in the certificate. It interacts with the License Component 380 to provide information about the animal owner for inclusion in the license.

The Certificate and License Access subsystem 400 uses the animal Owner Component 105 to obtain animal owner information either to verify it against some other identification device or to gain access to animal information for an animal that is owned by a particular owner.

The Appointment Scheduling subsystem 600 uses the animal owner component 105 to obtain owner information that is used to associate an animal owner with an appointment. The Appointment Component 605 is used to obtain the identity of the animal for which an appointment is to be scheduled. The animal Owner Component 105 also interacts with the Appointment Component 605 to provide information about itself during the process of scheduling an appointment.

### Animal Component 110

The animal component 110 defines the application's concept of an animal. An animal's properties, methods and access are controlled through the animal Component 110. Properties of an animal include data items that describe an animal or are used to, assist the application in processing Animal information. Examples of an animal's properties include: Animal name, Animal species, Animal breed, and Animal date of birth.

An animal method is software that manipulates Animal properties and manages the creation, deletion or updating of system Animal components stored either in memory, the database or both. Access to Animal properties and methods are performed in conjunction with the security components of the Registration subsystem 100.

The animal Component 110 is also used in the following subsystems: the Registration, Search and Recovery, Certificate Create, Certificate and License Access, Medical history, and Appointment Scheduling. The animal Component 110 is used extensively during the animal registration process. During this process, information pertaining to an animal is obtained from the user interface and stored in the system database by the animal Component 110. The animal Component 110 records associations to clinics, owners, DVMs, IDs, and other related entities. At the time of registration, the animal Component preferably interacts with the animal owner component 105 to supply a reference to itself for association purposes. The animal Component 110 interacts with the animal ID Component 120 to supply a reference to itself so that an animal ID may be used to find the associated Animal owner or Animal quickly. The animal Component 110 interacts with the animal Clinic Component 130 to supply a reference to itself for association purposes. It interacts with the DVM Component 140 to obtain a DVM reference to be stored with the animal record. Finally, the animal Component 110 interacts with the Audit Component 170 to log all changes to configuration information.

In the Search and Recovery subsystem 500, the animal Component 110 is used to obtain animal information helpful to create a missing animal message board thread and missing animal poster. The animal Component 110 interacts with the owner Notification, Search, and Recovery Component 505 to provide information about the lost animal that is used to create the missing animal message board thread. It interacts with the Contact Component 520 to provide information about the missing animal to forward onto the recipients of a missing animal e-mail. It interacts with the Missing Animal Poster 550 to provide information about the missing animal that will be used in the creation of a missing animal poster. The animal Component may interact with the animal ID Component 120 to supply a reference to itself which will be used then to gather information about the animal that will subsequently be used to report a missing animal.

The Medical history subsystem 200 uses the animal Component 110 to obtain animal information and to identify an animal that is to be referenced by a medical history item. The animal Component 110 interacts with the Medical History Component 205 to provide a reference to itself for the medical history record.

In the Certificate Create subsystem 300, the animal Component 110 interacts with the Certificate Component 305 to provide information about the animal for inclusion in a certificate. It interacts with the License Component 380 to provide information about the animal for inclusion in the license.

The Certificate and License Access subsystem 400 uses the animal Component 110 to gain access to the most current information pertaining to the animal. It is also used to identify the animal entry that will be used as a reference to look up certificates and licenses associated with that animal.

The Appointment Scheduling subsystem 600 uses the animal Component 110 to obtain a reference to an animal that is used to associate an animal with an appointment. The animal Component 110 interacts with the Appointment Component 540 to provide information about itself during the process of scheduling an appointment.

### Animal ID Component 120

The animal ID component 120 defines the application's concept of an animal ID. An animal ID's properties, methods and access are controlled through the animal ID Component 120. Properties of an animal ID are data items that describe an animal ID or are used to assist the application in processing Animal ID information. Examples of an animal ID's properties include: Animal ID number, Animal ID animal owner, Animal ID animal, and Animal ID status. Animal IDs are used to link animals with their owners.

An animal ID method is software that manipulates Animal ID properties and manages the creation, deletion or updating of system Animal ID components stored either in memory, the database or both. Access to Animal ID properties and methods are performed in conjunction with the security component.

The animal ID Component 120 is used during the Registration process to associate the animal owner, who is the owner of the animal ID, and the animal to which the ID is applied. The user interfaces may make use of the animal ID to identify a particular animal. Preferably each Animal ID stored in the system corresponds to an ID on a physical product, e.g., identification tag, card, or the like.

### Animal Clinic Component 130

This software component defines the application's concept of a clinic. A clinic's properties, methods and access are controlled through the animal Clinic Component 130. Properties of a clinic are data items that describe a clinic or are used to assist the application in processing clinic information. Examples of a clinic's properties include: clinic name, clinic address, clinic telephone number, and clinic enrollment status.

A clinic method is software that manipulates clinic properties and manages the creation, deletion or updating of system clinic components stored either in memory, the database or both. Access to clinic properties and methods are permitted based on the user ID permissions of the user.

The animal Clinic Component 130 interacts with many of the other system components in various subsystems including the Registration, Search and Recovery, Certificate Create, Medical history, and Appointment Scheduling subsystems to obtain information about the clinic required to perform the desired subsystem functionality. For the Registration subsystem 100, it may create a clinic entry in the system database if the clinic for which the animal being registered is not currently known to the system. The animal Clinic Component 130 uses the Audit Component 170 to record any configuration changes made to the clinic that are required to appear in the log file.

### DVM Component 140

This software component defines the application's concept of a Doctor of Veterinary Medicine (DVM). A DVM's properties, methods and access are controlled through the DVM Component 140. Properties of a DVM are data items that describe a DVM or are used to assist the application in processing DVM information. Examples of a DVM's properties include: DVM name, DVM license number, DVM telephone number, and DVM status.

A DVM method is software that manipulates DVM properties and manages the creation, deletion or updating of system DVM components stored either in memory, the database or both. Access to DVM properties and methods are permitted based on the user ID permissions of the user.

The Registration subsystem 100 uses the DVM Component 140 to either obtain an existing DVM entry that is to be associated with the newly registered animal or to create a DVM entry in the system database that is associated with an animal. The DVM Component 140 interacts with the animal Component 110 to associate each animal with the registered DVM. This association is established by default when an animal is registered through a registered DVM or clinic. Owners have the option to transfer the DVM association to another clinic and or DVM through the animal owner application interface. The DVM Component 140 uses the Audit Component 170 to record any configuration changes made to the DVM record that are required to appear in the log file.

The Certificate Create, Medical history, Appointment Scheduling, and Registration subsystems all use the DVM Component 140 as well. The Medical history subsystem 200 uses the DVM Component to obtain DVM information that is subsequently saved by the Medical history Item Component 205 for each clinic procedure performed by that DVM. The Certificate Create subsystem 300 uses the DVM Component 140 to obtain DVM information that the Certificate Component 305 records with the certificate.

Application Security Subgroup. Access to the registration components as well as other subsystem components is controlled by these security components within the registration subsystem 100. These are: the User Component 150, the Group Component 160, and the Audit Component 170. Registration activities are logged in a system audit file. Registered animal owners must supply a user name and a password that will be used in subsequent attempts to use the system.

### User Component 150

The User Component 150 defines an individual user's access to information contained in the system database. A user's properties, methods and access to information are controlled through the User Component. Properties of a user are data items that are used by the system in conjunction with the Group Component 160 to determine access to information. Examples of User Component properties include: login ID, password, time and date created, time and date when the login was last changed, keyword question and answer, status and user type. User type defines the user's functional need for information. Examples of user types include: clinic administrator, clinic employee, DVM, groomer employee, groomer administrator, police person, animal control officer, municipality employee and many others.

A User Component method is software that manipulates User Component properties to create or change login ID and password properties, to determine access rights based on the type user and user group of which a user is a member and authenticates the identity of the user.

The User Component 150 is invoked when a user logs into the system, at which time, relevant user information for that user is gathered and then used by the Registration, Certificate Create, Certificate and License Access, Medical history, Appointment Scheduling, and Search and Recovery subsystems. In the Registration subsystem 100, user account information is determined and set. In the Certificate Create and Certificate and License Access subsystems 300 and 400, the user information is used to constrain the activities of the user to those permitted for that user at that site. For example, an animal owner may view certificate information for an animal that they own, a Municipal User may view any animal's certificate, and a DVM may create a certificate. In the Appointment Scheduling subsystem 600, the information provided by this component is used to restrict activity for the user to those animals or sites associated with that user. For example, an animal owner will be able to schedule an appointment for an animal that belongs to them; and DVMs may view their appointments. In the Search and Recovery subsystem 500, the information provided by this component is used to permit users to the search and recovery activities allowed for that user. For example, an animal owner may report an animal as being lost if that animal belongs to that Animal owner. In the Medical history subsystem 200, this component grants permission to access or create Medical history Items based on the user ID.

### Group Component 160

The Group Component 160 defines the application's concept of an aggregation of users whose access to information is determined by shared membership in the group. Each user preferably will belong to one or more Groups. A Group is typically an organization entity and/or class to which a user belongs. Such an entity will typically be identified with a specific geographical address (physical facility) but may be defined as an entity that resides in multiple geographical addresses (physical facilities). In some cases a Group does not refer to an organization entity but instead defines a collection or class of individuals with a shared or common relationship with the system. An example of the latter is the animal owner group. Examples of other defined groups include DVM, System Administrator, Operator, Clinic User, Clinic Administrator, Municipal User, breeder, airline employee, groomer, etc. Group information is used to restrict user activity permitted for that type of user. For instance, Animal owners are not permitted to create certificates; and Clinic Administrators may be permitted to change a particular clinic's configuration.

A user's properties, methods and access to information are controlled through the Group Component 160. Properties of a Group are data items that are used by the application in conjunction with the User Component 150 to determine access to information. Examples of Group Component properties include: group name, date and time created, date of last change in group name, status, physical facility references and group type. Examples of Group types include: clinic, groomer, kennel, shelter, airline, international border customs, and breeder. A Group Component method is software that manipulates Group Component properties to create or change name, facility status and date properties.

This component is used when a user logs into the system, at which time, relevant group information for that user is gathered and then used by the Registration, Certificate Create and Certificate and License Access, Medical history, Appointment Scheduling, and Search and Recovery subsystems. In the Registration subsystem 100, group membership is determined and set. In the Certificate Create, Certificate and License Access, and Medical history subsystems, the group information is used to constrain the activities of the user to those permitted by that group of user. For example, DVMs may create certificates, Municipal Users may view certificate information for any animal, Clinic Users may create Medical history Items, DVMs may view Medical history Items, etc. In the Appointment Scheduling subsystem 600, the information provided by this component is used to restrict activity for the user to that permitted for the group to which the user belongs. For example, an animal owner will be able to schedule an appointment for an animal that belongs to them; DVMs may view their appointments; etc. In the Search and Recovery subsystem 500, the information provided by this component is used to permit users to the search and recovery activities allowed for that group.

### Audit Component 170

The Audit Component 170 records the use of other components and provides such information upon inquiry to system administrators and other's authorized by the system 99 with a need to know. The Audit Component 170 is used by several of the other components to record information to a log file that can later be reviewed if desired. Information to be logged is relative to the component that is using the Audit Component's services. For instance, the Certificate Component 300 uses the Audit Component 170 to log information pertaining to the creation of certificates. The License Component 380 uses the Audit Component 170 to record information pertaining to the creation of licenses. It is used by the animal owner component 105 to record the configuration activities of an animal owner. It is used by the animal Component 110 to record configuration changes made to an animal. It is used by the animal Clinic Component 130 to record configuration changes made to the clinic. It is used by the DVM Component 140 to record configuration changes made to a DVM entry. In addition to the above uses, the Audit Component 170 records information each time a user logs into and off of the system.

### Medical History Item Component 205

This software component defines the system's concept of a Medical History Item. A Medical History Item's properties, methods and access are controlled through the Medical History Item Component 205. Properties of a Medical History Item are data items that describe a Medical History Item or are used to assist the application in processing Medical History Item information. Examples of a Medical History Item's properties include: Medical History Item type, Medical History Item date, Medical History Item procedure, and Medical History Item vaccination.

A Medical History Item method is software that manipulates Medical History Item properties and manages the creation, deletion or updating of system Medical History Item components stored either in memory, the database or both. Access to Medical History Item properties and methods are performed in conjunction with the security component.

The Medical History Item Component 205 is used by the Certificate and License Creation and Medical History subsystems. The Medical History Item Component 205 interacts with many of the system components. A medical history item contains information about a clinic medical procedure that is performed on an animal. It interacts with the Animal Component 110 to obtain information and a reference to the animal on which the clinic procedure was performed. It interacts with the animal Clinic Component 130 to obtain information and a reference to the animal clinic that performed the clinic procedure. It interacts with the DVM Component 140, if the service provider was a DVM, to obtain DVM specific information that provided the service. It interacts with the Service Provider Component 610 to obtain information about the service provider. It interacts with the Clinic Procedure Type Component 210 to obtain information about the procedure performed on the animal. It interacts with the General Procedure Component 250 to obtain information about general clinic procedures (as opposed to vaccination procedures) that may be added to the clinic procedures and stored in the system database. It interacts with the User Component 150 to verify that the user has the necessary permission to create a medical record for the animal. Finally, it interacts with the Vaccination Procedure Component 255 to obtain specific information about the manufacturer of the vaccine, vaccine, and lot/serial number of the vaccine used in the vaccination.

The Medical History Item Component 205 is used by the Certificate Component 305 in the Certificate and License Creation subsystem to obtain information about the particular clinic procedure that was performed on an animal that is to be used in the creation of a certificate.

In the case where the clinic procedure performed was scheduled by the animal owner using the system software, the Medical History Item Component 205 will interact with the Appointment Component 605 to obtain a reference to the scheduled appointment.

### Clinic Procedure Type Component 210

This software component defines the application's concept of a Clinic Procedure. A Clinic Procedure's properties, methods and access are controlled through the Clinic Procedure Type Component 210. Properties of a Clinic Procedure Type are data items that describe a Clinic Procedure Type or are used to assist the application in processing Clinic Procedure Type information. Examples of a Clinic Procedure Type's properties include: name, duration, type, and status.

A Clinic Procedure Type method is software that manipulates Clinic Procedure Type properties and manages the creation, deletion or updating of system Clinic Procedure Type components stored either in memory, the database or both. Access to Clinic Procedure Type properties and methods are performed in conjunction with the security component.

The Clinic Procedure Type Component 210 is used in the Medical History subsystem 200. Clinic procedures as defined by the Clinic Procedure Type Component 210 provide the prototypes for each type of clinic procedure provided by the clinic. Clinic procedures may have a specific duration, name, status and service provider role associated with it. In addition, clinic procedures may contain information specific to its type.

When a Medical History Item is created, the system uses the Clinic Procedure Type as the basic building block to which it adds either Vaccination Procedure or General Procedure information. In other words, a clinic defines various Clinic Procedure Types; the service provider performs a type of clinic procedure on an animal, after which, a General Procedure 250 or Vaccination Procedure 255 entity is created using the Clinic Procedure Type 210 information.

### Clinic Procedures Components 215, Vaccination 220, Grooming 225, Other 230, Etc.

These components provide information to the Clinic Procedure Type Component 210 about their particular details upon which the Clinic Procedure Type Component 210 can build Clinic Procedure Type entry. These components are used in the Medical History subsystem 200.

### General Procedure Component 250

This component stores and manipulates all clinic procedures that are not vaccination procedures. All clinic procedures are grouped into one of two categories defined as general and vaccination procedures. General clinic procedures do not require that manufacturer, vaccine or lot information be stored in the system medical history item database. The General Procedure Component 250 assists the Medical History Item Component 205 in the storing of information about the animal, clinic, animal owner, clinic procedure, and DVM. This component is used in the Medical History subsystem 200.

### Vaccination Procedure Component 255

Clinic procedures that are defined as vaccination procedures require additional information to be stored in the system medical history item database as compared to the general clinic procedure. This additional information, for example, pertains to the manufacturer of the vaccine, the vaccine itself and the lot or serial number of the vaccine. This information is required for certification of rabies vaccinations. The Vaccination Procedure component 255 assists the Medical History Item Component 205 in the storage of vaccination type procedures performed on animals. This component is used in the Medical History subsystem 200.

### Lot Component 260

This software component stores vaccine lot information in the system database. Vaccine lot information includes manufacturing date, expiration date, lot/serial number, etc. Vaccination lot information that is retrieved from the database is handled by this component. This component is used in the Medical History subsystem 200.

### Vaccine Component 265

This software component stores vaccine information in the system database. Vaccine information includes product name, live/dead virus, life expectancy, etc. Vaccination information that is retrieved from the database is handled by this component. This component is used in the Medical History subsystem 200.

### Manufacturer Component 270

This software component stores vaccine manufacturer information in the system database. Manufacturer information includes manufacturer name, etc. Manufacturer information that is retrieved from the database is handled by this component. This component is used in the Medical History subsystem 200.

### Certificate Component 305

This software component defines the application's concept of a Certificate. A Certificate's properties, methods and access are controlled through the Certificate Component 305. Properties of a Certificate are data items that describe a Certificate or are used to assist the application in processing Certificate information. Examples of a Certificate's properties include: Certificate name, Certificate type, Certificate date, Certificate status, and Certificate clinic.

A Certificate method is software that manipulates Certificate properties and manages the creation, deletion or updating of system Certificate components stored either in memory, the database or both. Access to Certificate properties and methods are performed in conjunction with the security component.

The Certificate Component 305 is used in the Certificate and License Creation and Certificate and License Access subsystems 300 and 400. This component is used in the Certificate and License Creation subsystem 300 to create certificates and store them in the database. The data relating to a certificate preferably is a copy (not a reference) of the data obtained from their controlling components so that if any of the underlying data changes, the certificate data will remain constant. The Certificate Component 305 interacts with the animal Clinic Component 130 to obtain data about the clinic where the clinic procedure occurred. It interacts with the DVM Component 140 to obtain information about the DVM who performed the clinic procedure. It interacts with the animal Owner Component 105 to obtain data about the owner of the animal. It interacts with the Animal Component 110 to obtain information about the animal on which the clinic procedure was performed. It interacts with the Medical History Item Component 205 to obtain information about the clinic procedure performed on the animal of interest. It interacts with the User Component 150 to verify that the user has the necessary permission to create Certificates. The Certificate Component uses the Audit Component 170 to record the Certificate creation events.

Only authorized users may create certificates. These users are typically DVMs or their agents. Certificates are created using the application running on the clinic PC. Typically certificates are created for vaccination and spaying/neutering operations clinic procedures.

The Certificate Component 305 is used in the Certificate and License Access subsystem 400 to provide access to existing certificates and licenses. The Certificate Component 305 uses the Animal Component 110 to identify the animal whose certificates or licenses are to be viewed. It uses the animal Owner Component 105 to obtain current information from the system database about the animal owner. It interacts with the User Component 150 to verify that the user has the necessary permission to access a Certificate or License. The Certificate Component 305 uses the Audit Component 170 to record the Certificate access events.

Authorized users may view certificates. The animal owner may view all of the animal's certificates. Shelter employees, clinic employees, customs agents, kennel employees, groomers, municipal licensing agents, etc., also may be authorized to view current (non-expired) certificates stored in the system. The Certificate Component allows access to the stored certificate information based on the user ID access permissions.

### Health Certificate 310, Vaccination Certificate 320, Reproductive Certificate 330, USDA Travel Certificate Components 340

These components provide information to the Certificate Component 305 about each of the particular details upon which the Certificate Component 305 may build a Certificate. These components are used in the Medical History subsystem 200.

### License Component 380

This component is used in the Certificate and License Creation and Certificate and License Access subsystems 300 and 400. In the Certificate and License Creation subsystem 300, the License Component 380 interacts with the Animal Component 110 and Certificate Component 305 to create licenses. It uses the Animal Component 110 to gather all of the animal information necessary to create a license for a particular animal. It uses the Certificate Component 305 to determine whether the license should be granted or created. A municipal license issuance requires that an animal has been vaccinated against rabies. Municipalities also often offer reduced license fees if the animal has been spayed or neutered. Therefore the License Component 380 must interact with the Certificate Component 305 to verify that the certificate prerequisites for a particular license issuance have been met. It interacts with the User Component 150 to verify that the user has the necessary permission to create a license. The License Component 380 uses the Audit Component 170 to record the license creation events.

### Owner Notification, Search, and Recovery Component 505

This software component stores and retrieves, from the system database, pertinent information regarding registered lost animals. This component is used in the Search and Recovery subsystem 500. When animals are lost, appropriate information, e.g., information describing where and when they were last seen, may be stored in the database. The database is then updated with any new information that arrives involving the lost animal. Animal owners, system operators, system programs, “good Samaritans”, animal control officers, clinic personnel, etc. can all enter and search for information about a lost animal through the use of this component.

Animal owners, animal owner agents and system operators may report an animal as being lost. “Good Samaritans” may report an animal that they have found. To report a missing animal, an animal owner may call a toll free number or access a web site and create a missing animal report. The report is a form that may be filled out on-line with all of the pertinent information relating to the lost animal. The system then creates a message board thread for that lost animal. System activities and subsequent recovery action may be recorded in the system on the lost animal's message board thread. Preferably, the message board is viewable by anyone accessing the system, thus allowing anyone to post a message about the lost animal. Once the animal is returned to its owner, the message board thread for that lost animal is deleted and/or archived for an appropriate period of time.

“Good Samaritans” may report that they have found an animal by calling a toll free number or accessing a web site. If the animal has not yet been reported as missing, a new message board thread is created for the lost animal. The owner and the system operators are notified that an animal has been recovered. Arrangements may be made to return the lost animal to its owner using the message board thread, e-mail, though the system operators, or otherwise. Whichever method is selected depends on factors such as the information the “good Samaritan” makes known in the message board thread and the level of privacy the owner wishes to maintain. As one alternative, system operators may make arrangements with the “good Samaritan” to return the lost animal at a nearby clinic or shelter to preserve the anonymity of the animal owner.

### Contact Component 520

This component is used in the Search and Recovery subsystem 500. Certain lost animal entries entered into the system require that people and entities be notified electronically, e.g., via e-mail. This software component determines which entities and people should be notified and then sends out the appropriate communication to those recipients. Typically, system operators and animal owners are notified whenever an entry is made to the lost animal database. Other entities that may be contacted are animal shelters, animal clinics, kennels, police stations and the like. The contact component 520 keeps entries in the system database for all entities that are to be contacted. Such information, e.g., e-mail address, FAX number, contact telephone numbers, ZIP code, entity type, address, map, etc. are stored in the system database and made accessible through this component. Typically, entities are chosen for contact based on their proximity relative to the ZIP code where the animal was reported lost. Contact information for use to contact the animal owners and their agents is gathered from the animal owner component 100.

There are at least three basic types of electronic communications that preferably are sent out by this component: lost animal message, animal found message and informational messages. The lost animal message preferably contains the date, time and location where the animal was last seen. It also preferably contains a missing animal poster as an attachment; text with other descriptive information about the animal; and provisionally animal owner information. Privacy rules may apply which will prevent any or some of the owner information from being publicly disclosed. This type of electronic communication is sent to the system operators, animal owner, animal owner agent and the various entities within the prescribed ZIP code area.

The second type of electronic communication the system sends is the animal found message. This message preferably is sent to the animal owner, animal owner agent, system operators and the person/entity that reports that they have found the animal. Information contained in this e-mail preferably includes contact information date and time that the animal was found, and any other information that the finder of the animal may choose to enter into the system. When privacy rules require that the owner not be directly involved in the animal recovery, the system operator will arrange, with the animal finder, a convenient animal drop-off location.

The third type of electronic communication contains additional information about a lost animal. This information may be entered into the system by anyone who may have spotted the missing animal and wishes to report the sighting. It may also be additional information that the animal owner may wish to convey about their animal that may help locate it or care for it once it is found but before it is returned. Finally, the electronic communication may contain update information designed to keep the animal owner, system operators, and the public current with respect to the status of the search and recovery operation currently in progress for a particular animal. Search status update information may be entered by the system or the system operators.

### Missing Animal Poster Component 550

This component is preferably used in the Search and Recovery subsystem 500. The missing animal poster component 550 creates poster of missing animals. These posters may be printed locally on a user's printer. They also may be automatically created by the system and sent electronically to certain entities whenever an animal is reported missing. The missing animal posters preferably contain a picture of the missing animal; pertinent animal information; the date when the animal was lost; the location of where the animal was last seen; and when privacy rules permit, owner and owner contact information. Animal owners and animal owner agents may create missing animal posters of the animals on the provided Internet web site.

### Appointment Component 605

This software component defines the application's concept of an Appointment. Appointment's properties, methods and access are controlled through the Appointment Component 605. Properties of an Appointment are data items that describe an Appointment or are used to assist the application in processing Appointment information. Examples of an Appointment's properties include: Appointment date, Appointment type, Appointment service provider, and Appointment status.

An Appointment method is software that manipulates Appointment properties and manages the creation, deletion or updating of system Appointment components stored either in memory, the database or both. Access to Appointment properties and methods are performed in conjunction with the security component.

The Appointment Component 605 is used in the Appointment Scheduling subsystem 600. This component is used when appointments are to be scheduled. Scheduling an appointment requires the interaction between this component and many others. The Appointment Component 605 interacts with the animal owner component 105 to obtain a reference to the owner entry and information about the owner of the animal for which an appointment is to be scheduled. It interacts with the animal Component 110 to obtain information about the animal for which the appointment is to be scheduled. It interacts with the animal Clinic Component 130 to obtain a reference to the clinic entry at which the appointment is to be scheduled. The Appointment Component 605 interacts with the Appointment Type Component 640 to obtain a reference to the type of appointment to be scheduled. The Appointment Component 605 interacts with the Service Provider Component 610 to obtain information about the person who is scheduled to perform the service on the animal.

### Service Provider Component 610

This software component defines the application's concept of a Service Provider. A Service Provider's properties, methods and access are controlled through the Service Provider Component 610. Properties of a Service Provider are data items that describe a Service Provider or are used to assist the application in processing Service Provider information. Examples of a Service Provider's properties include: Service Provider name, Service Provider type, Service Provider ID, Service Provider status, and the like.

A Service Provider method is software that manipulates Service Provider properties and manages the creation, deletion or updating of system Service Provider components stored either in memory, the database or both. Access to Service Provider properties and methods are performed in conjunction with the security component.

The Service Provider Component 610 is used in the Appointment Scheduling and Medical history Items subsystems 200 and 600. It interacts with the animal Clinic Component 130 to provide Service Provider references for those Service Providers associated with the animal Clinic. The Service Provider Component 610 interacts with the Availability Component 620 to provide Service Provider references for which availability may be defined and determined. It interacts with the Role Component 550 to obtain a reference to the type of Role that the Service Provider plays during the execution of the appointment. It interacts with the Appointment 540 and Medical history Item 520 Components in the corresponding component sections.

### Availability Component 620

This software component defines the application's concept of Availability for service providers and clinics. The Availability Component's properties, methods and access are controlled through the Availability Component 620. Properties of the Availability Component are data items that describe availability or are used to assist the application in processing availability information. Examples of the Availability Component's properties include: availability date, time, recurrence indicator, and status.

An Availability Component method is software that manipulates properties and manages the creation, deletion or updating of system properties stored either in memory, the database or both. Access to Availability properties and methods are performed in conjunction with the security component.

The Availability Component 620 is used in the Appointment Scheduling subsystem. It interacts with the animal Clinic Component 130 to provide information and references pertaining to the animal Clinic's availability for appointment scheduling purposes. It interacts with the Service Provider Component 610 to provide references to pertaining to the Service Providers' availability for appointment scheduling purposes.

### Role Component 630

This software component defines the application's concept of a Service Provider's Role. A Role's properties, methods and access are controlled through the Role Component 630. Properties of a Role are data items that describe a Role or are used to assist the application in processing Role information. Examples of a Role's properties include: Role name, Role type, and Role status.

A Role method is software that manipulates Role properties and manages the creation, deletion or updating of system Role components stored either in memory, the database or both. Access to Role properties and methods are performed in conjunction with the security component.

The Role Component 630 is used in the Appointment Scheduling subsystem 600. It interacts with the Appointment Type Component 640 to provide information about the roles of service providers. When appointments are scheduled, the type of appointment will dictate the type or role of the service provider required. For example, if the appointment is for a grooming, then the service provider that is to provide the service must have a role of groomer. This component also interacts with the Service Provider Component 610 as mentioned in that section.

### Appointment Type Component 640

The Appointment Type Component 640 is used in the Appointment Scheduling subsystem 600. When an animal owner desires to schedule an appointment for an animal, the owner may choose from a list of different appointment types. Each appointment type preferably has a name and a number of time units associated with it. Each appointment type preferably also indicates what role a service provider will fulfill in order to satisfy the appointment.

This component interacts with the Time Unit Component 650 to obtain the defined time unit length. It also interacts with the animal Clinic Component 130 to provide references to the various defined Appointment Types. It interacts with the Role Component 630 to obtain Role definitions and it provides information to the Role Component 630 regarding the Role requirements of the various defined Appointment Types.

### Time Unit Component 650

The Time Unit Component 650 is used in the Appointment Scheduling subsystem 600. It provides and defines the value for a unit of time. Each Appointment Type duration is expressed in time units. The Appointment Component 605 uses the duration as defined by the Time Unit Component 650 to determine the duration of a scheduled appointment and determine availability.

A Time Unit entry is associated with each defined Animal Clinic 130 entry because each clinic may determine what the value of the time unit for their clinic should be. The Time Unit Component 650 also interacts with the Appointment Component 605 as defined in the Appointment Type Component 640.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

What is claimed is:

1) A multiuser system for storing, accessing, and verifying animal information, said system comprising:
i) a database controllably accessed by a plurality of user classes from multiple locations;
ii) a plurality of identification codes stored in the database, each of said codes being uniquely associated with a single animal; and
iii) animal data stored in the database, said data comprising information relating to a plurality of animals, said data being associated with corresponding animals by information comprising a plurality of respective identification codes, and wherein said data comprises certified information indicative of a government-regulated status of corresponding animals.

2) A multiuser system for storing, accessing, and verifying animal information, said system comprising:
i) a database;
ii) a plurality of identification codes stored in the database, each of said codes being associated with an animal;
iii) animal data stored in the database, wherein said system comprises security rights that control at least accessing, editing, and authoring the animal data, said rights being established based upon information comprising:
1. at least one of the identification codes;
2. author information indicative of the authorship of the data;
3. a class characteristic indicative of a class to which the data belongs; and
iv) information indicative of whether at least a portion of the data comprises a certified, government-regulated status.

3) A multiuser system comprising a, database comprising certified and verifiable information about a plurality of animals, wherein the database is remotely accessible by a plurality of classes of users, each class of user being subject to a corresponding security that controllably allows one or more user classes to access the database and at least one of (i) verify animal information or (ii) determine a certified status of animal data.

4) A multiuser system for storing, accessing, and verifying animal information, said system comprising a database comprising legal status information for a plurality of animals, said database comprising:
i) official, electronically stored, government authorized data corresponding to a plurality of rabies vaccinations for a plurality of animals; and
ii) official, electronically stored animal license data, wherein said license data is dependent from information comprising corresponding, electronically stored rabies data.

5) A multiuser system for storing, accessing, and verifying animal information, said system comprising
i) a database:
ii) official, electronically stored, government authorized data stored in the database, said data comprising certified information indicative of the legal status of a plurality of animals; and
iii) a security that controllably allows one or more user classes to access the database in order to verify a legal status of at least one animal.

6) A method of creating an electronic record indicative of the rabies vaccination status of an animal, comprising the steps of:
i) accessing a multiuser system comprising a database comprising electronically stored animal information;
ii) determining that the animal satisfies a criteria for obtaining a rabies vaccination certification;
iii) storing electronic health information in the database, said health information comprising rabies vaccination information for the animal; and
iv) storing certified information indicative of the rabies vaccination status of the animal, said certified information being electronically linked to the rabies vaccination information.

7) A method of creating electronic, certified information indicative of the license status of an animal, comprising the steps of:
i) accessing a multiuser system comprising a database, said database comprising certified information indicative of the rabies vaccination status for the animal;
ii) determining that the animal satisfies a criteria for obtaining a license from information comprising the certified information;
iii) creating electronic, certified license information for the animal;
iv) storing the electronic, certified, license information in the database.

8) A method of documenting a rabies vaccination for an animal, comprising the steps of:
i) administering a rabies vaccination to an animal;
ii) accessing a multiuser system comprising a database;
iii) causing certified information to be stored in the database, said certified information relating to a rabies vaccination status for the animal;
iv) creating a tangible rabies vaccination element, said element comprising information relating to the rabies vaccination status for the animal and said element comprising information relating to the remote database;
v) attaching the tangible rabies vaccination element to the animal.

9) An animal information system, comprising:
i) a multiuser database comprising electronically stored animal information, said information comprising:
1. a plurality of animal identification codes, each of said codes being associated with a corresponding animal;
2. electronically stored, certified rabies vaccination information for a plurality of vaccinated animals, said information being associated with an animal by information comprising a corresponding animal identification code; and
3. electronically stored, certified license information for a plurality of licensed animals, said information being associated with a licensed animal by information comprising a corresponding animal identification code; and
(b) at least one of (1) a first tangible fixture attached to an animal that comprises rabies vaccination information, and said element further comprising information relating to the database; or (2) a second tangible fixture attached to an animal that comprises license information, and said element further comprising information that relates to the database.

10) A method of caring for an animal, comprising the steps of:
i) administering a health treatment to an animal;
ii) storing health information relating to the treatment in a multiuser system comprising a database, said system comprising a security that controllably allows one or more user classes to access the database to verify a legal status of at least one animal; and
iii) storing certified information relating to the health treatment in the database, wherein the certified information is created under an authority comprising a governmental body and wherein the certified information is dependently linked to the health information.

11) A method of caring for an animal, comprising:
i) administering a health treatment to an animal;
ii) causing the treated animal to be registered in a multiuser system, said registration comprising the step of associating a unique animal identification code with the animal;
iii) storing health information relating to the treatment in the database, said health information being associated with the animal by information comprising the unique animal identification code; and
iv) using information comprising the stored health information to perfect a government authorized status for the animal, said status being documented by certified information stored in the database and said status being associated with the animal by information comprising the unique animal identification code.

12) A multiuser system for managing animal information, said system comprising a multiuser database comprising animal-related information, said animal information comprising:
i) stored health information for an animal comprising information indicating that the animal has had a health treatment; and
ii) certified, government authorized information stored in the database, said government authorized information comprising information indicative of compliance by the animal with a governmental rule, wherein a prerequisite for complying with said rule includes said health treatment, and wherein the certified government authorized, information is dependently linked to the health information.

13) A method of caring for an animal, comprising the steps of:
i) providing a database comprising animal related information for a plurality of animals, at least a portion of said information having an expiration date;
ii) generating a report comprising at least portions of the information and at least one corresponding expiration date;
iii) using information comprising at least a portion of the report to cause a follow-up action to be taken with respect to an animal having an expiration date included on the report.

14) An animal identification card, comprising
i) information relating to a characteristic of the animal;
ii) information comprising an image of the animal; and
iii) information relating to a system comprising a database, wherein data relating to the animal is electronically stored.
